(19) United States
(12) Patent Application Publication
(10) Pub. No.: US 2026/0218216 A1
(43) Pub. Date: Jul. 30, 2026

(54) Flow Guiding Barrel and Shoot Apical Meristem Delivery System

(71) Applicants: Hermes Biomaterials Inc., Ames, IA (US); University of Maryland, College Park, College Park, MD (US)

(72) Inventors: Connor Thorpe, Ankeny, IA (US); Weifeng Luo, College Park, MD (US); Yiping Qi, College Park, MD (US); Shan Jiang, Ames, IA (US)

(21) Appl. No.: 19/395,425

(22) Filed: Nov. 20, 2025

Related U.S. Application Data
(60) Provisional application No. 63/723,389, filed on Nov. 21, 2024.

(51) Int. Cl. C12N 15/82

(52) U.S. Cl. CPC C12N 15/8207

(57) ABSTRACT

A system, method, and apparatus for delivering genetic material to a shoot apical meristem. The delivery system may have a biolistic particle delivery assembly having a gas acceleration tube and a bombardment chamber. The bombardment chamber has a microcarrier launch assembly comprising an opening adapted to receive gas for acceleration of the microcarrier system and to direct gas to a target. The delivery system may have a source of pressurized carrier gas operatively connected to a first end of the gas acceleration tube; and an extender. The extender has a channel configured to fit through the opening of the microcarrier launch assembly. The length of the extender facilitates the penetration of the microcarrier system to an L2 layer of the shoot apical meristem, conferring germline transformation and/or modification.

### CROSS-REFERENCE TO RELATED APPLICATIONS

The present application claims priority to U.S. Provisional Application 63/723,389 titled A Flow Guiding Barrel and Shoot Apical Meristem Delivery System filed on Nov. 21, 2024, the entirety of which is hereby incorporated by reference herein, including any figures, tables, drawings, and other information.

### SEQUENCE LISTING

The instant application contains a Sequence Listing which has been submitted in XML format via EFS-Web and is hereby incorporated by reference in its entirety. Said XML copy is named Hermes Biomaterials Sequence Listing.xml, created on Mar. 30, 2026 and is 30 kilobytes in size.

### FIELD OF THE DISCLOSURE

This disclosure relates generally to genetic material delivery in shoot apical meristems. More specifically and without limitation, this disclosure relates to using a delivery system to control the penetration of genetic material in the L2 layer of cells in shoot apical meristems.

### OVERVIEW OF THE DISCLOSURE

Genetic engineering in plants is a large field with enormous potential. However, the current biolistic particle delivery system used in this field suffers from low efficiency (<1%) when it is used for shoot apical meristem (“SAM”) delivery. To improve the performance of the current system, it is common to perform three repeated bombardments to increase the percentage of genetic material successfully delivered to shoot apical meristems. However, performing repeated bombardments is time consuming and expensive. In addition, repeated bombardments will induce more cell damage and cause undesirable chromosomal damages to the shoot apical meristems. Further, the current biolistic delivery system used in the genetic engineering field yields highly inconsistent results, which causes time and resource waste. Furthermore, there is no easy way to control the depth of penetration for the current biolistic particle delivery system.

The present disclosure describes a new way to deliver a reagent deeper into the plant tissue through a biolistic delivery device or gene gun. This is very significant for SAM delivery, since only when the delivery reaches to the deeper layer (L2 layer) of the SAM, can the transgene or targeted genome modifications be transmitted to next generations. As described herein, a Flow Guiding Barrel (“FGB”) device or extender was added to a biolistic particle delivery device to facilitate deeper penetration of genetic material into the L2 layer using a relatively low pressure of carrier gas. In one particular example, DNA encoded with a Cas12a protein sequence is introduced to SAM. Once expressed, the Cas12a will be generated and edit the target gene(s) with the guide RNA (gRNA). As such, the FGB allows gold particles to efficiently penetrate deeper into the L2 layer of the SAM, enabling stable transformation of the plant to the following generations. Without FGB, it usually takes the conventional system three bombardments to reach the L2 layer along with low efficiency, a higher gas pressure, and more cellular damage. With FGB, only one bombardment is needed. Therefore, utilizing the FGB leads to a higher ratio of stably integrated events carried onto the successive generation of a plant than previous methods.

Therefore, for all the reasons stated above, and the reasons stated below, there is a need in the art for a genetic material delivery system that improves upon the state of the art.

Another object of the disclosure is to provide a genetic material delivery system that provides improved functionality over prior art systems.

Yet another object of the disclosure is to provide a genetic material delivery system that provides improved features over prior art systems.

Another object of the disclosure is to provide a genetic material delivery system that is relatively inexpensive.

Yet another object of the disclosure is to provide a genetic material delivery system that is easy to use.

Another object of the disclosure is to provide a genetic material delivery system that is intuitive to use.

Yet another object of the disclosure is to provide a genetic material delivery system that is strong and robust.

Another object of the disclosure is to provide a genetic material delivery system that can be used in many applications.

Yet another object of the disclosure is to provide a genetic material delivery system that improves efficiencies.

Another object of the disclosure is to provide a genetic material delivery system that provides unique functionality.

Yet another object of the disclosure is to provide a genetic material delivery system that is fast to use and fast to set-up.

Another object of the disclosure is to provide a genetic material delivery system that is safe to use.

Yet another object of the disclosure is to provide a genetic material delivery system that saves time.

Another object of the disclosure is to provide a genetic material delivery system that is high quality.

These and other objects, features, or advantages of the disclosure will become apparent from the specification, figures and claims.

### BRIEF DESCRIPTION OF THE FIGURES

FIG. 1 depicts a system of delivering genetic material to SAM using the disclosed delivery system in accordance with one or more arrangements.

FIG. 2 depicts another view of a system of delivering genetic material to SAM using the disclosed delivery system in accordance with one or more arrangements.

FIG. 3A depicts a genetic material delivery system without an extender or FGB in accordance with one or more arrangements.

FIG. 3B depicts a genetic material delivery system with an extender or FGB in accordance with one or more arrangements.

FIG. 4 depicts a tabletop genetic material delivery system in accordance with one or more arrangements.

FIG. 5 depicts a tabletop genetic material delivery system in accordance with one or more arrangements, the view showing a control system along with a gas regulator for use in delivery of genetic material to SAM.

FIG. 6 depicts a handheld genetic material delivery system in accordance with one or more arrangements.

FIG. 7A depicts a conventional genetic material delivery system as well as the disclosed genetic material delivery system, the view showing the difference in SAM penetration between the conventional genetic material delivery system and the disclosed genetic material delivery system.

FIG. 7B depicts the difference in material delivery between a conventional genetic material delivery system and the disclosed genetic material delivery system, the figure showing that the disclosed genetic material delivery system is more efficient and effective at delivery of material to SAM.

FIG. 8 is a side elevation view of an extender or FGB in accordance with one more arrangements.

FIG. 9 is a front perspective view of an extender or FGB in accordance with one or more arrangements.

FIG. 10 is another front perspective view of an extender or FGB in accordance with one or arrangements, the view indicating the diameter of the opening of the extender or FGB.

FIG. 11 is a top perspective view of an extender or FGB in accordance with one or more arrangements.

FIG. 12A is a top elevation view of an extender or FGB in accordance with one or more arrangements.

FIG. 12B is a bottom elevation view of an extender or FGB in accordance with one or more arrangements.

FIG. 13 is another side elevation view of an extender or FGB in accordance with one or more arrangements.

FIG. 14 is another front perspective view of an extender or FGB in accordance with one or arrangements, the view showing dividers that create multiple flow paths for the microcarrier system described herein.

FIG. 15 is a top perspective view of a placement guide configured to facilitate the introduction of genetic material or proteins into plants, in accordance with one or more arrangements.

FIG. 16 is a side elevation view of a placement guide configured to facilitate the introduction of genetic material or proteins into plants, in accordance with one or more arrangements.

FIG. 17A depicts the difference in material delivery between a conventional genetic material delivery system and the disclosed genetic material delivery system which includes an extender or FGB, the figure showing differences in particle distribution of such material.

FIG. 17B depicts the difference in material delivery between a conventional genetic material delivery system and the disclosed genetic material delivery system which includes an extender or FGB, the figure showing that material delivered using the disclosed genetic material delivery system results in a high level of material being delivered to SAM.

FIG. 18A depicts the difference in material delivery between a conventional genetic material delivery system and the disclosed genetic material delivery system which includes an extender or FGB, the figure showing an alternative image depicting that material delivered using the disclosed genetic material delivery system results in a high level of material being delivered to SAM.

FIG. 18B depicts the difference in material delivery between a conventional genetic material delivery system and the disclosed genetic material delivery system which includes an extender or FGB, the figure showing differences in penetration depth and target area between the conventional genetic material delivery system and the disclosed genetic material delivery system.

FIG. 19A depicts the depth of penetration of the disclosed genetic material delivery system when used to deliver material to SAM in accordance with one or more arrangements.

FIG. 19B depicts the difference in material delivery between a conventional genetic material delivery system and the disclosed genetic material delivery system which includes an extender or FGB, the figure showing differences in penetration depth and amount of material delivered between the conventional genetic material delivery system and the disclosed genetic material delivery system.

FIG. 20 depicts the disclosed genetic material delivery system having increased GE efficiency over conventional genetic material delivery systems.

FIG. 21 depicts the disclosed genetic material delivery system having increased GFP efficiency over conventional genetic material delivery systems.

FIG. 22A depicts the TaPDS gene, further identified by SEQ ID NO: 3, which was used to test GE efficiency and GFP efficiency of the disclosed genetic material delivery system.

FIG. 22B depicts the TaPDS gene including the ttLbCas12a and crRNA, both driven by the ZmUbi promoter.

### SUMMARY OF THE DISCLOSURE

In one or more arrangements, as shown for example, a delivery system for delivering genetic material (DNA, RNA, protein, or a combination thereof) to a shoot apical meristem is disclosed herein. Such delivery system includes a biolistic particle delivery assembly, a source of pressurized carrier gas operatively connected to a first end of a gas acceleration tube, and an extender. The biolistic particle delivery assembly has a gas acceleration tube and a bombardment chamber with a microcarrier launch assembly having an opening adapted to receive gas for acceleration of the microcarrier system and to direct gas to a target. The extender has a channel configured to fit through the opening of the microcarrier launch assembly system. A length of the channel runs from a top end of the extender to a bottom end of the extender and is configured to direct a major flow path of the microcarrier system through the channel and towards the target cells. The length of the channel facilitates the penetration of the microcarrier system to an L2 layer of the shoot apical meristem.

In one or more arrangements, as shown for example, a delivery system for delivering genetic material to shoot apical meristems includes a biolistic particle delivery assembly configured to deliver a microcarrier system at target cells of the shoot apical meristem. The biolistic particle delivery system having a gas tube and a cartridge holder having an opening. The delivery system includes a source of pressurized carrier gas operatively connected to a first end of the gas tube. The delivery system includes an extender having a channel configured to fit through the opening of the cartridge holder. The channel is configured to direct a major flow path of the microcarrier system through the channel and towards the target cells. A length of the extender facilitates the penetration of the microcarrier system to an L2 layer of the shoot apical meristem.

In one or more arrangements, as shown for example, an extender has a ring with a top end, a bottom end, and at least one side. The ring has an opening configured to receive a microcarrier system configured to edit a genome of a plant. The bottom of the ring is configured to attach the extender to a biolistic particle delivery assembly. The extender has a body attached to the ring having a top end, a bottom end, and at least one side. The body has a channel configured to receive pressurized carrier gas carrying the microcarrier system towards target cells of the plant. A length of the extender facilitates the penetration of the microcarrier system to an L2 layer of the shoot apical meristem.

In one or more arrangements a method for delivering genetic material to a shoot apical meristem includes attaching an extender to a biolistic particle delivery assembly and placing a microcarrier system in the biolistic particle delivery assembly. The method also includes moving pressurized gas through a gas tube from a gas container to the biolistic particle delivery assembly and bombarding the microcarrier system though the extender towards target cells utilizing the pressurized gas through the extender; and penetrating an L2 layer of a shoot apical meristem by the microcarrier system.

### DETAILED DESCRIPTION OF THE DISCLOSURE

In the following detailed description of the embodiments, reference is made to the accompanying drawings which form a part hereof, and in which is shown by way of illustration specific embodiments in which the disclosure may be practiced. The embodiments of the present disclosure described below are not intended to be exhaustive or to limit the disclosure to the precise forms in the following detailed description. Rather, the embodiments are chosen and described so that others skilled in the art may appreciate and understand the principles and practices of the present disclosure. It will be understood by those skilled in the art that various changes in form and details may be made without departing from the principles and scope of the invention. It is intended to cover various modifications and similar arrangements and procedures, and the scope of the appended claims therefore should be accorded the broadest interpretation so as to encompass all such modifications and similar arrangements and procedures. For instance, although aspects and features may be illustrated in or described with reference to certain figures or embodiments, it will be appreciated that features from one figure or embodiment may be combined with features of another figure or embodiment even though the combination is not explicitly shown or explicitly described as a combination. In the depicted embodiments, like reference numbers refer to like elements throughout the various drawings.

It should be understood that any advantages and/or improvements discussed herein may not be provided by various disclosed embodiments, or implementations thereof. The contemplated embodiments are not so limited and should not be interpreted as being restricted to embodiments which provide such advantages or improvements. Similarly, it should be understood that various embodiments may not address all or any objects of the disclosure or objects of the invention that may be described herein. The contemplated embodiments are not so limited and should not be interpreted as being restricted to embodiments which address such objects of the disclosure or invention. Furthermore, although some disclosed embodiments may be described relative to specific materials, embodiments are not limited to the specific materials or apparatuses but only to their specific characteristics and capabilities and other materials and apparatuses can be substituted as is well understood by those skilled in the art in view of the present disclosure.

It is to be understood that the terms such as “left, right, top, bottom, front, back, side, height, length, width, upper, lower, interior, exterior, inner, outer, and the like as may be used herein, merely describe points of reference and do not limit the present invention to any particular orientation or configuration.

As used herein, the term “or” includes one or more of the associated listed items, such that “A or B” means “either A or B”. As used herein, the term “and” includes all combinations of one or more of the associated listed items, such that “A and B” means “A as well as B.” The use of “and/or” includes all combinations of one or more of the associated listed items, such that “A and/or B” includes “A but not B,” “B but not A,” and “A as well as B,” unless it is clearly indicated that only a single item, subgroup of items, or all items are present. The use of “etc.” is defined as “et cetera” and indicates the inclusion of all other elements belonging to the same group of the preceding items, in any “and/or” combination(s).

As used herein, the singular forms “a,” “an”, “and” “the” are intended to include both the singular and plural forms, unless the language explicitly indicates otherwise. Indefinite articles like “a” and “an” introduce or refer to any modified term, both previously-introduced and not, while definite articles like “the” refer to a same previously-introduced term; as such, it is understood that “a” or “an” modify items that are permitted to be previously-introduced or new, while definite articles modify an item that is the same as immediately previously presented. It will be further understood that the terms “comprises,” “comprising”, “includes”, and/or “including”, when used herein, specify the presence of stated features, characteristics, steps, operations, elements, and/or components, but do not themselves preclude the presence or addition of one or more other features, characteristics, steps, operations, elements, components, and/or groups thereof, unless expressly indicated otherwise. For example, if an embodiment of a system is described as comprising an article, it is understood the system is not limited to a single instance of the article unless expressly indicated otherwise, even if elsewhere another embodiment of the system is described as comprising a plurality of articles.

It will be understood that when an element is referred to as being “connected”, “coupled”, “mated,” “attached,” “fixed,” etc. to another element, it can be directly connected to the other element, and/or intervening elements may be present. In contrast, when an element is referred to as being “directly connected”, “directly coupled”, “directly engaged” etc. to another element, there are no intervening elements present. Other words used to describe the relationship between elements should be interpreted in a like fashion (e.g., “between” versus “directly between”, “adjacent” versus “directly adjacent”, “engaged” versus “directly engaged,” etc.). Similarly, a term such as “operatively”, such as when used as “operatively connected” or “operatively engaged” is to be interpreted as connected or engaged, respectively, in any manner that facilitates operation, which may include being directly connected, indirectly connected, electronically connected, wirelessly connected or connected by any other manner, method or means that facilitates desired operation. Similarly, a term such as “communicatively connected” includes all variations of information exchange and routing between two electronic devices, including intermediary devices, networks, etc., connected wirelessly or not. Similarly, “connected” or other similar language particularly for electronic components is intended to mean connected by any means, either directly or indirectly, wired and/or wirelessly, such that electricity and/or information may be transmitted between the components.

It will be understood that, although the ordinal terms “first,” “second,” etc. may be used herein to describe various elements, these elements should not be limited to any order by these terms unless specifically stated as such. These terms are used only to distinguish one element from another; where there are “second” or higher ordinals, there merely must be a number of elements, without necessarily any difference or other relationship. For example, a first element could be termed a second element, and, similarly, a second element could be termed a first element, without departing from the scope of example embodiments or methods.

Similarly, the structures and operations discussed herein may occur out of the order described and/or noted in the figures. For example, two operations and/or figures shown in succession may in fact be executed concurrently or may sometimes be executed in the reverse order, depending upon the functionality/acts involved. Similarly, individual operations within example methods described below may be executed repetitively, individually or sequentially, to provide looping or other series of operations aside from single operations described below. It should be presumed that any embodiment or method having features and functionality described below, in any workable combination, falls within the scope of example embodiments.

As used herein, various disclosed embodiments may be primarily described in the context of a genetic material delivery system. However, the embodiments are not so limited. It is appreciated that the embodiments may be adapted for use in various other applications, which may be improved by the disclosed structures, arrangements and/or methods. The system is merely shown and described as being used in the context of a genetic material delivery system for ease of description and as one of countless examples.

### System 10:

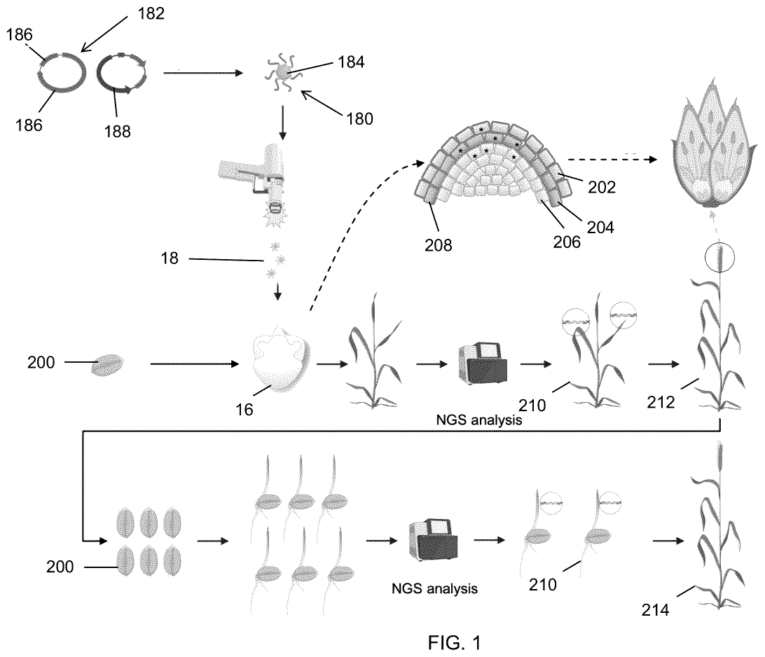

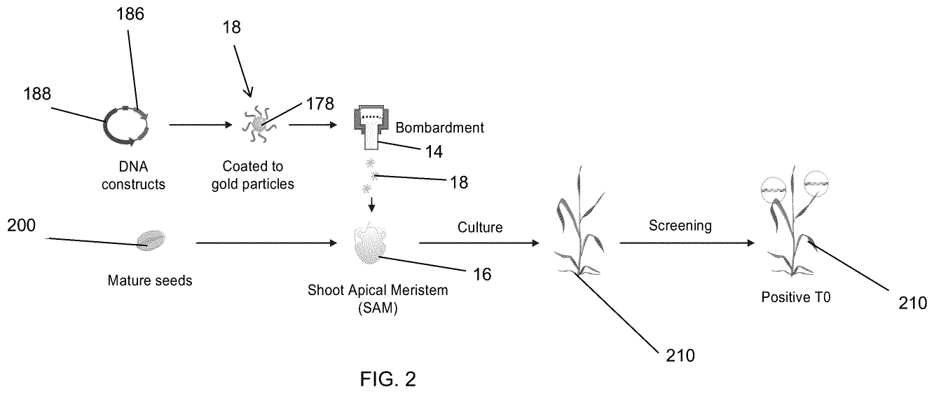

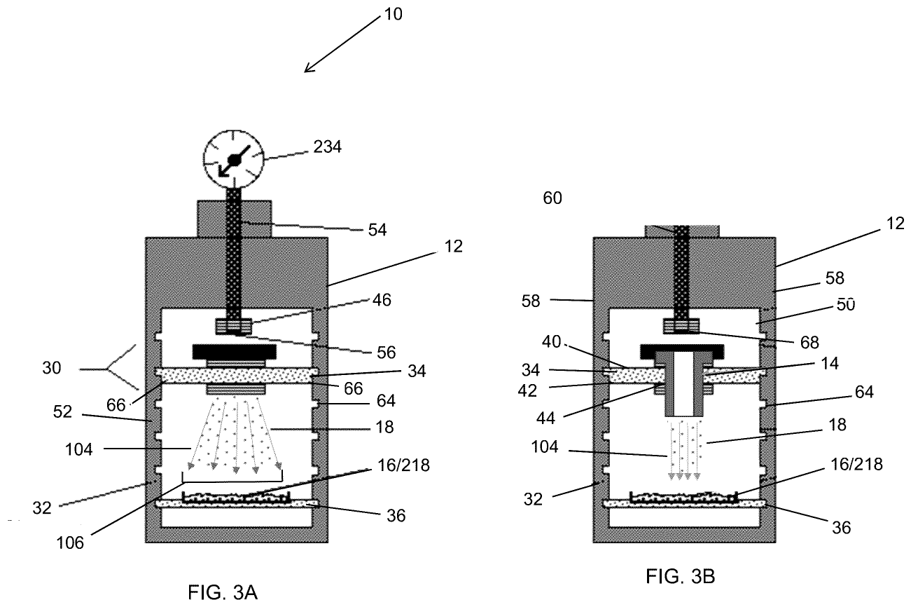

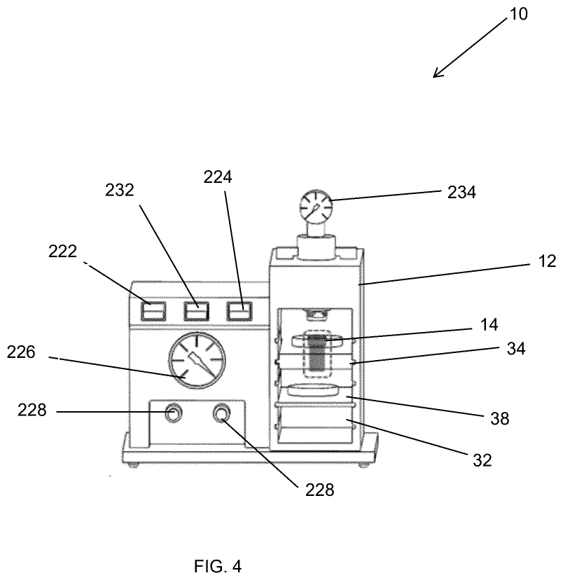

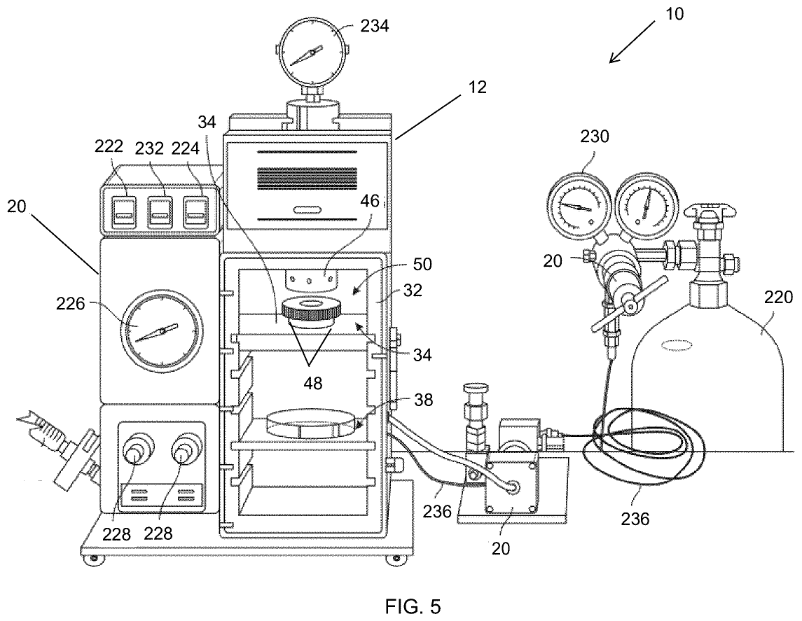

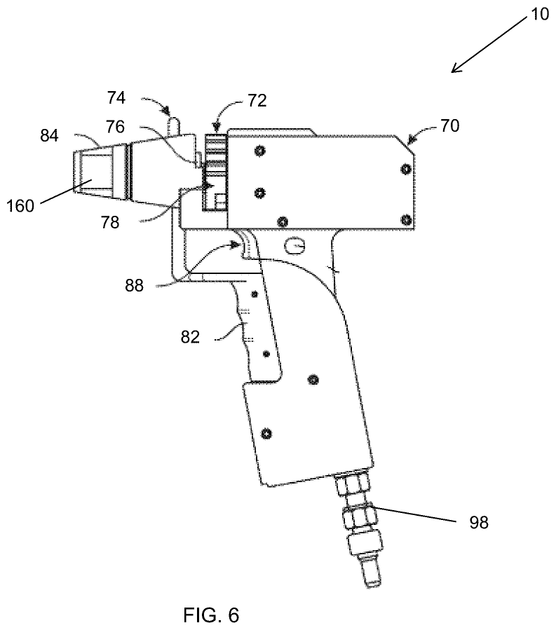

With reference to the figures, a genetic material delivery system 10 (or simply system 10) is presented. The genetic material delivery system 10 is formed of any suitable size, shape and design and is configured to facilitate the precise delivery of genetic material 186 to the target cells 218 of the SAM 16 or control the penetration of the bombardment of genetic material at the target cells 218 of the SAM 16, as illustrated in FIGS. 1 and 2. In the arrangement shown, as one example, system 10 includes a biolistic particle delivery assembly 12, an extender 14, the SAM 16, a microcarrier system 18, and a control system 20, among other components.

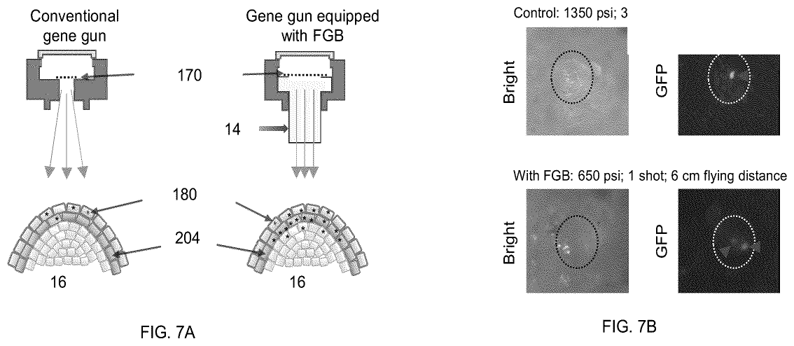

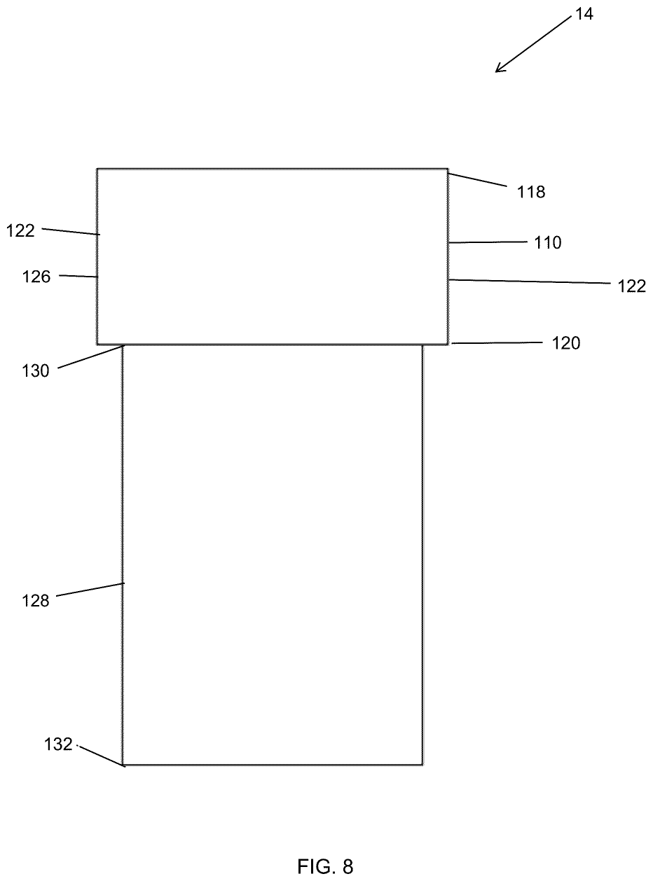

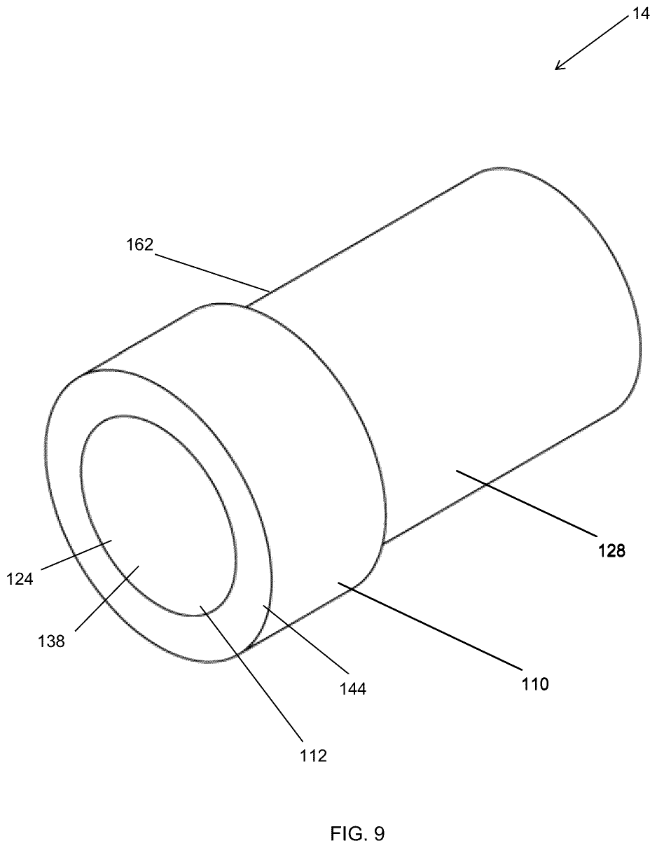

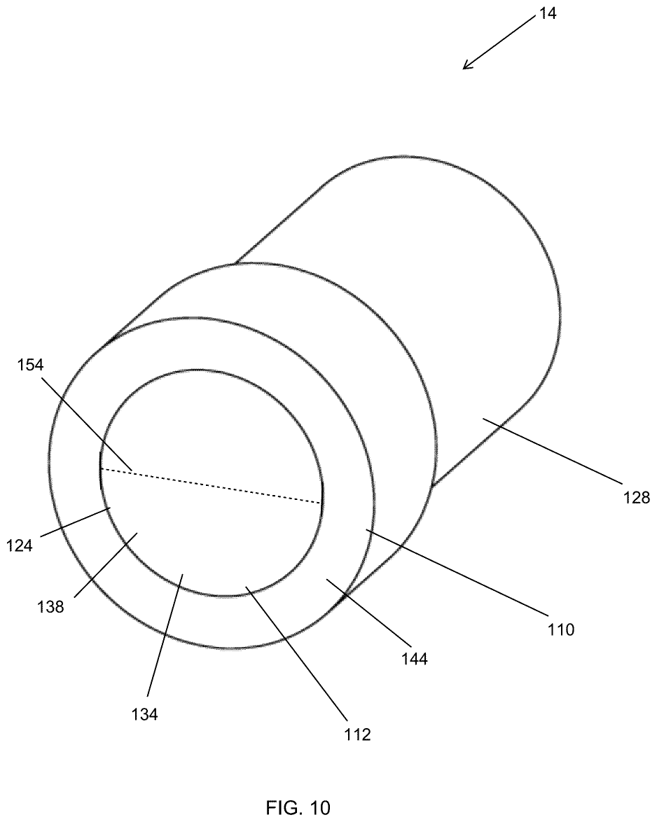

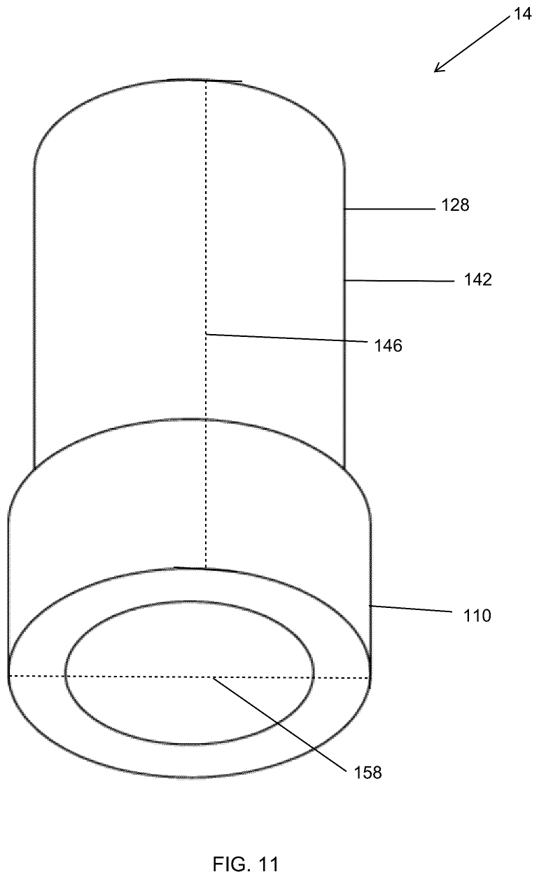

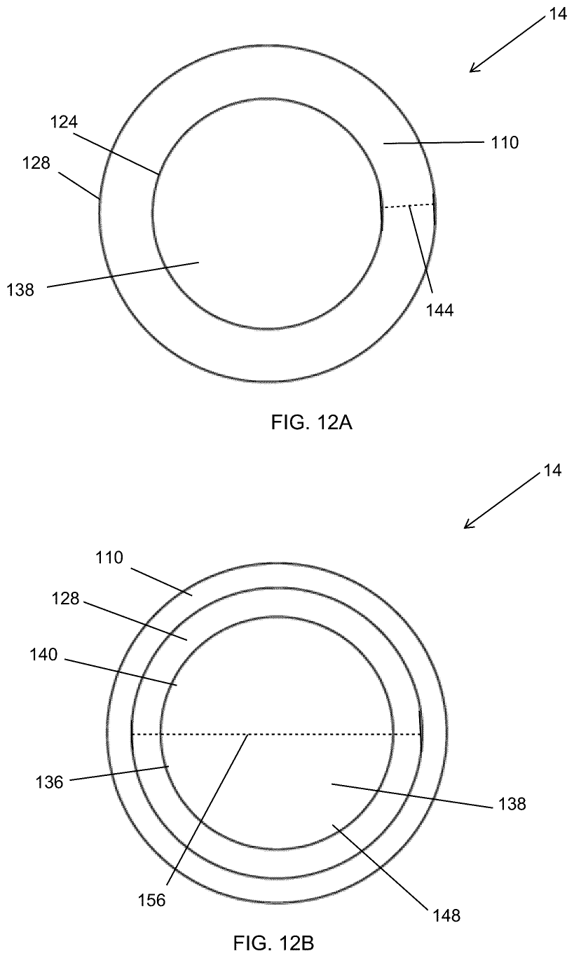

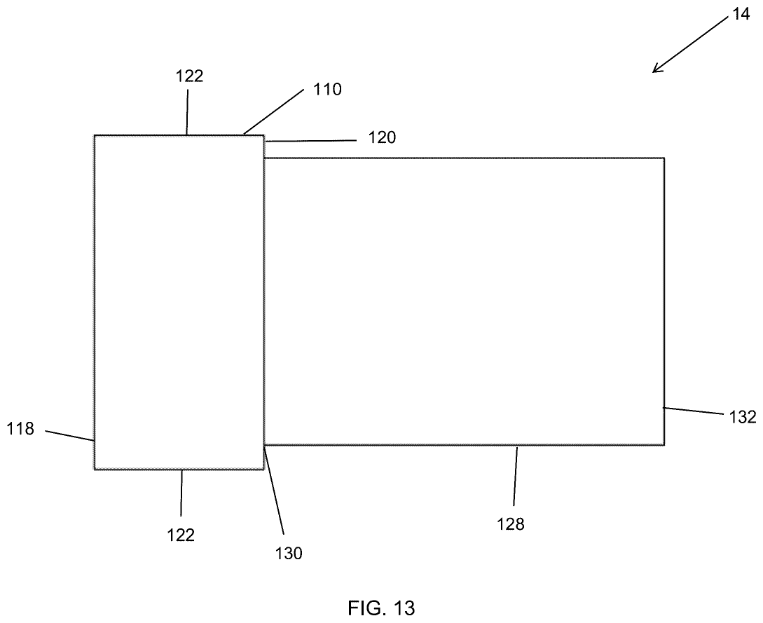

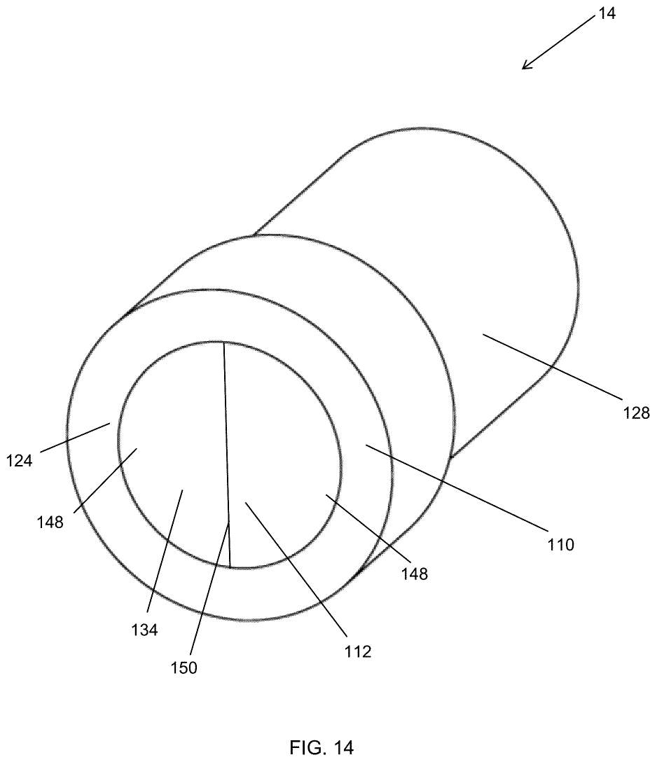

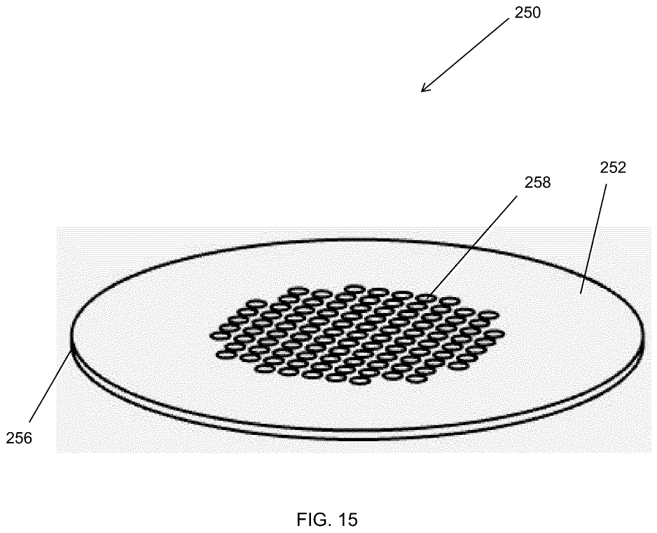

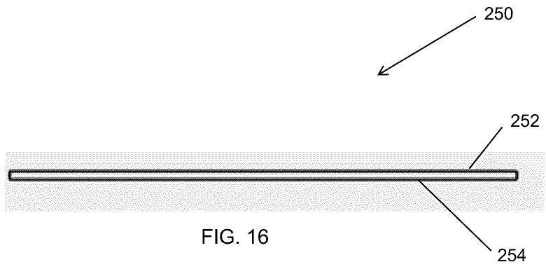

System 10 provides a method to deliver genetic material 186 and proteins 188 into SAM 16 by utilizing a flow guide barrel (“FGB” or extender 14). The extender 14 is a simple attachment to the biolistic particle delivery assembly 12 or the Bio-Rad PDS/He 1000 biolistic particle delivery system 12. In one or more arrangements, as shown for example, the penetration of the genetic material 186 and proteins 188 via a microcarrier system 18 was controlled by adjusting the pressure and particle flight distance or flying distance 104 when such materials were delivered by a biolistic particle delivery assembly 12 with the enablement of the extender 14. Such activity collimates in the delivery of the genetic material 186 or proteins 188 into the germline lineage cells 208 present in the L2 layer 204 of the SAM 16 in plants 210, as illustrated in FIG. 7A.

### Shoot Apical Meristem 16:

The terms plant 210 or seed 200 as used herein encompass natural plants and seeds and artificial plants and seeds. The plant 210 or seed 200 may refer to any plant from the kingdom Plantae or angiosperms including flowering plants, cereal grains, grain legumes, grasses, roots and tuber crops, vegetable crops, fruit plants, pulses, medicinal crops, aromatic crops, beverage plants, sugars and starches, spices, oil plants, fiber crops, latex crops, food crops, feed crops, plantation crops or forage crops.

A fully matured seed 200 refers to a seed 200 that comes into full maturity where the process of maturing after pollination is completed. The SAM 16 gives rise to the above ground plant 210 body, including the leaves, flowers, stems and auxiliary branches. The SAM 16 is a multicellular tissue containing a stem cell niche, which activity allows for a dynamic equilibrium between maintenance of germ cells 208 and production of differentiated cells that are incorporated in new aerial tissues and lateral organs produced in the SAM 16. The germ cell 208 encompasses germ cells ranging from a primordial germ cell, to an oocyte and a spermatoblast. SAM 16 is divided into distinct cell layers termed: L1 layer cells 202/epidermal, L2 layer cells 204/subepidermal and L3 layer cells 206/corpus cell layer. During sexual reproduction, only one layer, the L2 layer cells 204 contribute to the next generation by forming gametes. L2 layer cells 204 of the SAM 16 will develop into germ cells 208, thereby transmitting the edited genome to the subsequent generations 214 from the parent generation 212.

### Biolistic Particle Delivery Assembly 12:

The biolistic particle delivery assembly 12 is formed of any suitable size, shape and design and is configured to facilitate the introduction of genetic material 186 into plants 210. In the arrangement shown, as one example, the biolistic particle delivery assembly 12 includes microcarrier launch assembly 30, chamber 32, target cell shelf 36, and a gas acceleration tube 54, among other components. In one or more arrangements the biolistic particle delivery assembly 12 may be the PDS-1000/He instrument, which is manufactured and sold by Bio-Rad Laboratories (Hercules, CA; hereinafter “Bio-Rad”). However, this arrangement is not meant to be so limited. In another arrangement, as shown, for example, biolistic particle delivery assembly is a portable gene gun 70 or another other size, shape and design configured to facilitate the introduction of genetic material 186 into plants 210.

### Chamber 32:

Chamber 32 is formed of any suitable size, shape, and design and is configured to facilitate the introduction of genetic material 186 into plants 210. Chamber 32 has a top section 50 and a bottom section 52 separated by the microcarrier launch assembly 30 and opposing sides 58. The opposing sides 58 of chamber 32 have a plurality of recesses configured to receive opposing sides 66 of the microcarrier launch assembly 30, allowing the microcarrier launch assembly 30 to be raised or lowered in the chamber 32. The top section 50 of chamber 32 has a gas acceleration tube 54 configured to facilitate the entry of pressured gas 60 into chamber 32 through an opening 68 at the chamber end of the gas acceleration tube 54. A rupture disk retaining cap 46 seals the rupture disk 56 against the opening 68 of the gas acceleration tube 54. The rupture disk retaining cap 46 is tightened securely until it bursts due to pressurized gas 60 in the gas acceleration tube 54. Chamber 32 has a door allowing a user to access one or more components of the chamber 32, for example so that they may place the microcarrier system 18 on the microcarrier assembly shelf 34. However, this arrangement is not meant to be limited. Rather it is understood that chamber 32 may have size, shape and design to facilitate the bombardment of genetic material 186 or proteins 188 at the target cells 218.

### Microcarrier Launch Assembly 30:

The microcarrier launch assembly 30 is formed of any suitable size, shape, and design and is configured to facilitate the introduction of genetic material 186 into plants 210. The microcarrier launch assembly 30 has a launch assembly shelf 34 having a top 40 opposing a bottom 42, opposing sides 66, and an opening to facilitate the microcarrier system 18 prior to bombardment of genetic material 186 or proteins 188 at the target cells 218. The microcarrier launch assembly 30 has an opening 44, having a diameter 48, configured to facilitate the bombardment of microcarrier system 18 comprising the genetic material 186 or proteins 188 towards the target cells 218. The microcarrier launch assembly 30 may separate a top section of chamber 50 from the microcarrier assembly shelf 34 of chamber 32 from a bottom section of chamber 52. The microcarrier launch assembly 30 holds the microcarrier system 18 in preparation for the bombardment of the microcarrier system 18. However, this arrangement is not meant to be limited. Rather it is understood that the microcarrier launch assembly 30 may have any size, shape, and design to facilitate the bombardment of genetic material 186 or proteins 188 at the target cells 218.

In one or more arrangements, as shown for example, microcarrier system 18 has a stop screen 170. The microcarrier system 18 is halted after a short distance by the stop screen 170. The stop screen 170 is formed of any suitable size, shape, and design and is configured to separate macrocarriers 190 (not shown) from microcarriers 180. Stop screen 170 may be made from stainless steel, metal, plastic, or different materials. The microcarriers 180 continue traveling toward the target cells 218 to penetrate and transform the target cells 218. The stop screen has a top 176 (not shown) opposing a bottom 192 (not shown) and sides 168 (not shown). The stop screen has a width 172 (not shown) or diameter and a height 174 (not shown) to facilitate the separation of the microcarriers 180 from the macrocarriers 190 (not shown). In one or more arrangements, a diameter of stop screen 170 is sized and shaped to fit within extender 14, the interior 124 of retaining ring 110, or an interior 140 of body 128. In one or more arrangements as shown, the stop screen 170 may have one or more openings sized to allow the microcarriers 180 to pass through but which are also small enough to prevent the macrocarriers 190 (not shown) from traveling through. For example, the wire diameter of the openings of the stop screen 170 can be 0.3-0.6 mm with opening size of 0.7-0.9 mm, as long as the stop screen 170 can sustain the impact of any carriers and effectively stop the movement of the macrocarriers 190 (not shown). In other arrangements, for example, the diameter of the stop screen 170 may be larger or smaller. Further, in one or more arrangements, for example, the opening size may be larger or smaller. However, this arrangement is not meant to be limited. Rather it is understood that the stop screen 170 may have any size, shape, and design to facilitate the bombardment of genetic material 186 or proteins 188 at the target cells 218.

### Target Shelf 36:

The target shelf 36 is formed of any suitable size, shape, and design and is configured to facilitate the introduction of genetic material 186 into plants 210. The target shelf 36 holds the biological target cells 218 of the SAM 16 in a container 38, such as a petri plate in the path of the accelerated DNA/microcarrier preparation. Particle flight distance 104 is determined by positioning the target shelf 36 at one or more levels using recesses 64 in the chamber sides 58 to adjust the particle flight distance 104 and particle flight width 106 of the microcarrier system 18. The bombardment process utilizes high-pressure gas 60 released by a rupture disk 56, and partial vacuum (e.g., gas acceleration tube 54) to propel the microcarrier system 18 loaded with microscopic microcarriers 180 toward target cells 210 at a high velocity. However, this arrangement is not meant to be limited. Rather it is understood that the target shelf 36 may have any size, shape, and design to facilitate the bombardment of genetic material 186 or proteins 188 at the target cells 218.

The target shelf 34 may have an embryo or seed 200 placement guide 250 formed of any suitable size, shape, and design and that is configured to facilitate the introduction of genetic material 186 or proteins 188 or a combination thereof into plants 210, seeds 200, or SAM 16. The placement guide 250 has a top 252, an opposing bottom 254, and at least one side 256. The placement guide 250 may have one or more holes 258 to place the seeds 200 or embryos in prior to bombardment. The placement guide 250 may be in, above, or underneath a petri dish. In one or more arrangements the placement guide 250 may be placed directly on target shelf 36.

However, this arrangement is not meant to be limited. Rather it is understood that the placement guide 250 may have any size, shape, and design to facilitate the bombardment of genetic material 186 or proteins 188 at the target cells 218.

### Portable Gene Gun 70:

In the arrangement shown, as one example, a biolistic particle delivery assembly 12 includes portable gene gun 70 having cartridges 72, cartridge holders 78, a trigger button 88, a power source 96 (not shown), and gas attachment 98, among other components, such as a gas acceleration tube 54. The portable gene gun 70, may be comprised of, for example, a Helios Gene Gun. The Helios Gene Gun is manufactured and sold by Bio-Rad. The portable gene gun 70 may not require a vacuum and any target cell 218 accessible to the barrel 160 can be transformed. Consequently, the portable gene gun 70 may be used in a much wider variety of gene transfer applications and provides a tool for both in vitro and in vivo transformations. Essentially, any type of cells which can be made accessible to its barrel 160 may be transformed.

In one or more arrangements as shown, for example, portable gene gun 70 has one or more cartridges 72 which are configured to hold the microcarrier system 18 which enters the target cells by the gas 60. A cylinder lock 74 controls movement of the barrel pin 76. The cylinder lock 74 is positioned in the backward locked position so that the barrel pin 76 is inserted into the hole in the cartridge holder 78 to keep the cartridge holder 78 in a proper position for firing or bombarding the microcarrier system 18. The cylinder lock 74 may be moved forward and to the right to a firing position and this will disengage the barrel pin 76 from the cartridge holder 78. The cartridge holder 78 may be removed from the portable gene gun 70, but only after compressing the cylinder advance lever 82, to prevent any damage to the O-rings 80 (not shown). The cylinder advance lever 82 is activated by pulling it backwards.

The cylinder advance lever 82 is released when a cartridge 72 is inserted or removed and the cylinder advance lever 82 moves the barrel liner 84 backward, bringing the O-ring 80 (not shown) on the back of the barrel liner 84 in contact with the cartridge holder 78. After discharging microcarrier system 18 from one cartridge 72, pulling the cylinder advance lever 82 moves the barrel 160 forward to increase the space for inserting the cartridge holder 78 behind the barrel liner 84. This ratchets the cartridge holder 78, bringing the next cartridge 72 into firing position. The trigger button 88 controls the flow of helium gas through the portable gene gun 70. The trigger button 88 acts as a switch and momentarily permits helium to enter the cartridge 72 and barrel 160. A gas attachment 98 facilitates the attachment of portable gene gun 70 to a hose.

However, this arrangement is not meant to be limited. Rather it is understood that the portable gene gun 70 may have any size, shape, and design to facilitate the bombardment of genetic material 186 or proteins 188 at the target cells 218.

### Extender 14:

Extender 14 (or FGB) is formed of any suitable size, shape and design and is configured to facilitate precise delivery and penetration of microcarrier system 18 to the target cells 218 of the L2 layer 204. In the arrangement shown, as one example, extender 14 includes a retaining ring 110 and a body 128, among other components. In one or more arrangements, system 10 may have multiple extenders 14 of the same shape and size or various shapes and sizes.

### Retaining Ring 110:

Retaining ring 110 is formed of any suitable size, shape, and design and is configured to facilitate attaching extender 14 to the biolistic particle delivery assembly 12. In one or more arrangements, for example, as shown, retaining ring 110 has a top 118, a bottom 120, and one or more sides 122. Retaining ring 110 has at least one opening 112 to permit the microcarrier system 18 to travel from the top 40 of microcarrier launch assembly shelf 34 through the opening 44 of microcarrier launch assembly shelf 34 and towards the target cells 218. Microcarrier system 18 travels through an interior 124 of the retaining ring 110. Interior 124 of retaining ring 110 opposes exterior 126 of retaining ring 110. Retaining ring 110 may be removably attached to body 128 or permanently connected to body 128. Retaining ring 110 and opening 112 of retaining ring 110 may have a circular shape or any other shape configured to facilitate attaching extender 14 to the biolistic particle delivery assembly 12. The length 146 of the extender 14 can vary, however in some embodiments, is at least four times greater than the thickness 144 of the retaining ring 110.

Retaining ring 110 acts as a head of extender 14. Retaining ring 110 has a larger diameter 158 than that of the body 128 so that the body 128 does not slide through the openings 44 of the microcarrier assembly shelf 34 or an opening of the cartridge holder 78. Bottom 120 of retaining ring 110 rests on the top 40 of the microcarrier assembly shelf 34 and the body 128 is inserted through opening 44 of the microcarrier assembly shelf 34.

However, this arrangement is not meant to be limited. Rather it is understood that the retaining ring 110 may have any size, shape, and design to facilitate the bombardment of genetic material 186 or proteins 188 at the target cells 218.

### Body 128:

Body 128 is formed of any suitable size, shape and design and is configured to facilitate the control of the penetration and precise delivery of genetic material 186 or proteins 188 to the target cells 218. In one or more arrangements, for example, as shown, body 128 has a top 130, a bottom 132, and one or more sides 162. Body 128 has an interior 140 opposing an exterior 142. Top 130 of body 128 has one or more openings 134 and bottom 132 has one or more openings 136. Channel 138 may connect opening 134 to opening 136. Channel 138 permits the microcarrier system 18 to travel from the top 40 of microcarrier launch assembly shelf 34 through the microcarrier launch assembly shelf 34 and towards the target cells 218.

Body 128 is configured to be inserted into opening 44 of the microcarrier assembly shelf 34 and has a diameter 156 that is smaller than opening 44. Body 128 and channel 138 may have a circular shape or any other shape configured to control the penetration and delivery of microcarrier system 18. The diameter 154 of channel 138 may be the same diameter of opening 112 of retaining ring 110. In other arrangements, for example the diameter 154 of channel 138 may be larger or smaller than the diameter of opening 112 of retaining ring 110. Opening 134 of top 130 of body 128 may have a larger or smaller diameter than opening 136 of bottom 132 of body 128, giving the channel 138 a tapered shape.

Channel 138 provides one or more flow paths 148 for the microcarrier system 18 to travel through during bombardment of the target cells 218. Channel 138 may have one or more dividers 150 to create multiple flow paths 148 for the microcarrier system 18 to travel. The particle flight width 106 of the flow paths 148 of the microcarrier system 18 may be based upon a length 146 of the extender 14 and particle flight distance 104.

In one or more arrangements, for example, the extender 14 may have a stop screen holder, not shown, configured to secure the stop screen 170 to the body 128 or the retaining ring 110 of the extender 14.

However, this arrangement is not meant to be limited. Rather it is understood that the body 128 may have any size, shape, and design to facilitate the bombardment of genetic material 186 or proteins 188 at the target cells 218.

### Control System 20:

Control system 20 is formed of any suitable size, shape, and design and is configured to facilitate the introduction of genetic material 186 into plants 210. In the arrangement shown, as one example, control system 20 includes gas containers 220, a power mechanism 222, a vacuum switch 224, a vacuum gauge 226, a vacuum rate valve 228, a vacuum control valve 230, a gas flow switch 232, and a gas pressure gauge 234, among other components.

In one or more arrangements, as shown, for example, power mechanism 222 controls the supply of electrical power to system 10. Power mechanism 222 may be an on/off switch, a button, a regulator, a valve or any other mechanism that can control the supply of electrical power to system 10. Gas flow switch 232 controls the flow of carrier gas, such as helium, from gas container 220, through gas tube 236, to the gas acceleration tube 54. Gas container 220 is configured to store the carrier gas. The gas may be pressurized. Gas pressure gauge 234 measures the pressure of the carrier gas in the gas acceleration tube 54. The gas flow switch 232 may be held on continuously until rupture disk 56 bursts and then is released to stop the flow of carrier gas. Vacuum switch 224 is configured to control a vacuum operatively attached to chamber 32 to facilitate the movement of the microcarrier system 18 to the target cells 218. The vacuum gauge 226 is configured to indicate a level of vacuum in chamber 32. Vacuum rate 228 is configured to regulate rate of the flow of filtered air from the vacuum in chamber 32.

In one or more arrangements as shown, for example, portable gene gun 70 is connected to a gas container 220 by gas tubing 236 using gas flow switch 232. Gas attachment 98 of portable gene gun 70 facilitates the connection of portable gene gun 70 to gas tubing 236.

However, this arrangement is not meant to be limited. Rather it is understood that the control system 20 may have any suitable size, shape, and design to facilitate the bombardment of genetic material 186 or proteins 188 at the target cells 218.

### Microcarrier System 18:

Microcarrier system 18 is formed of any suitable size, shape, and design and is configured to facilitate the introduction of genetic material 186 into plants 210. In the arrangement shown, as one example, control system 20 includes microcarriers 180, plasmids 182, a coating 184, genetic material 186, proteins 188, reagents, or macrocarriers 190 (not shown), among other components. In one or more arrangements, for example, the microcarrier system 18 may have one or more samples 178. Each sample 178 may include a microcarrier 180, a plasmid 182, a coating 184, a macrocarrier 190 (not shown), and genetic material 186 or protein 188 or a combination thereof, among other components. Samples 178 may have the same components or different components. If there are a plurality of samples 178, the samples 178 may have the same microcarriers 180, plasmids 182, coatings 184, macrocarriers 190 (not shown) and reagents or one or more of these components may be different between the samples 178. For example purposes only, a first sample may include genetic material 186 with plasmid 182 while a second sample 178 may include a protein 188 with plasmid 182. A single bombardment may comprise a plurality of samples 178 of microcarrier system 18. Microcarriers 180 are coated with genetic material 186, proteins 188 or other biological material for transformation. In one or more arrangements, the microcarriers 180 may be attached to macrocarriers 190 (not shown) to facilitate transport of microcarriers 180 towards the target cells 218. Macrocarriers 190 (not shown) may be halted after a short distance by a stop screen 170 or other mechanism. The microcarriers 180 will then continue traveling toward the target cells 218 and penetrate the target cells 218.

The plasmid 182 may be comprised of genetic material 186 or proteins 188 and is attached to the microcarriers 180. The microcarriers 180 may have a coating 184, such as a gold coating, tungsten coating, other metal coating, a natural coating, or a synthetic coating. To deliver microcarriers 180 into L2 layer cells 204, the optimal penetration depth for microcarriers 180 should be between 20 μm and 35 μm, considering the L1 layer cells 202 are approximately 20 μm thick. Delivery efficiency can be enhanced by optimizing bombardment conditions to deliver the microcarriers 180 into L2 layer cells 204.

Plasmid 182 may be attached to the microcarrier 180 of the genetic material 186 or proteins 188 from a solution in the presence of gold coated 184 microcarriers 180 and polycarbon spermidine by the addition of CaCl₂). The microcarriers 180 are then washed extensively with ethanol to remove the water and resuspended in ethanol. In one or more arrangements the microcarrier 180 solution is coated onto the inner wall of tubing such as gold coated tubing, and dried. The tubing may be cut into cartridges 72 and inserted into portable gene gun 70.

However, this arrangement is not meant to be limited. Rather it is understood that the microcarrier system 18 may have any suitable size, shape and design to facilitate the bombardment of genetic material 186 or proteins 188 at the target cells 218.

### In Operation:

In operation, system 10 accelerates microcarriers 180 via a burst of pressurized gas 60 (e.g., helium, nitrogen, etc.) from gas acceleration tube 54 or through the top of the portable gene gun 70. The microcarriers 180 are sent through the barrel 160 or through the microcarrier assembly shelf 34 and toward plant tissue having the target cells 218. The length 146 and diameter 154 of channel of the extender 14 are critical factors to control precise delivery of the microcarriers 180 to the L2 layer cells 204. After the microcarriers 180 are loaded into biolistic particle delivery assembly 12, a burst of pressurized gas flows through the biolistic particle delivery assembly 12 and the confinement of the extender 14 reduces turbulence in the chamber 32. The major flow path 148 within the channel 138 is therefore substantially laminar causing less damage to the target cells 218 and a decrease in particle flight width 106 of the microcarrier system 18 during bombardment. In one or more arrangements, a single bombardment may comprise one or more samples 178. In another arrangements, for example, the SAM 16 may receive multiple bombardments, each bombardment carrying a different sample 178.

In one or more arrangements, system 10 may operate as described by the following example. Seeds were sterilized in a 50% bleach solution for 10 minutes, rinsed with sterile distilled water, and placed in Petri dishes lined with autoclaved filter paper for overnight germination at room temperature. Using a needle (32G 4 mm; Medt, USA) under a microscope, the coleoptile and leaf primordia covering the SAM 16 were carefully removed. Embryos were excised from the endosperms and cultured on osmotic Murashige and Skoog (MS) medium containing 20 g/L sucrose, 0.98 g/L MES (pH 5.8), 36.4 g/L D-sorbitol, 36.4 g/L D-mannitol, 3% plant preservative mixture (Nacalai Tesque, Japan), and 7.0 g/L phytagel (Sigma-Aldrich, USA). Fifty embryos were placed per plate for bombardment. GFP fluorescence was assessed 12 hours post-bombardment using a fluorescence microscope (ZEISS) with a GFP filter.

The embryos may be transferred to normal MS medium (without D-sorbitol and D-mannitol) and incubated at 30° C. for 5 days, followed by transfer to a growth chamber (16 hours light/8 hours darkness, at 22° C.). After three weeks, plants were transferred to soil and grown under the same long-day conditions (16 hours light/8 hours darkness, 22° C.).

In one or more arrangements, a ZmUbi_GFP vector and ttLbCas12a_crRNA_SAM all-in-one vector were constructed and employed. In the ZmUbi_GFP vector, the GFP gene was driven by the ZmUbi promoter. In the ttLbCas12a_crRNA_SAM vector, ttLbCas12a was driven by the Ubi promoter, while the crRNA was expressed using a ribozyme system, also driven by the Ubi promoter.

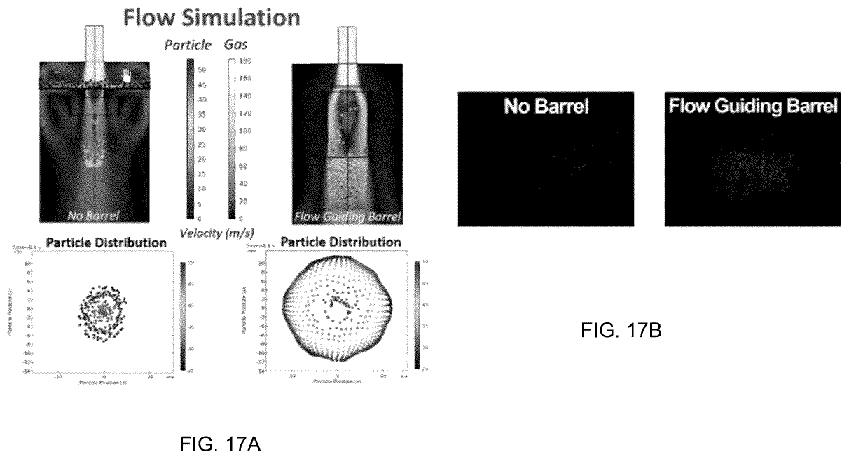

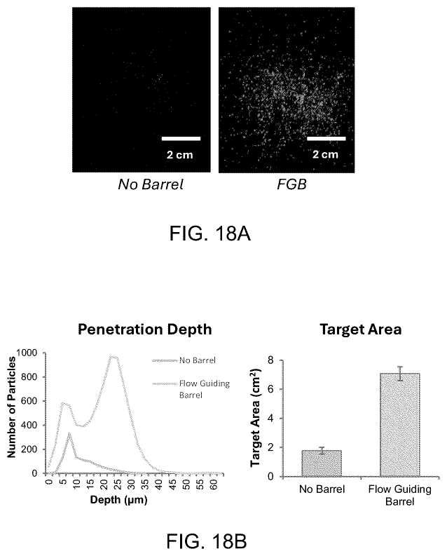

One of the keys to enhance genetic material delivery efficiency is to effectively deliver the gold coated particles containing the genetic material 186 or proteins 188 into the deeper L2 layer 204. Delivery that cannot reach to L2 layer 204 will not be effective. That is one major reason the conventional biolistic particle delivery assembly does not work effectively since there are no effective ways to control penetration of the bombardment into the L2 layer 204 when using the conventional system. Based on computational fluid dynamics (CFD) simulations and experimental validation in agarose gels, installing a custom designed extender 14 to the biolistic particle delivery assembly 12 will significantly enhance the delivery range and depth in plant 210 tissues. This predicted outcome has been confirmed by the delivery of fluorescein isothiocyanate-conjugated bovine serum albumin (FITC-BSA) loaded gold particles into the 1 wt. % agaros gel mimicking tissue strength and tracking the particles via confocal microscopy, as shown in FIGS. 17A, 17B, and 18A.

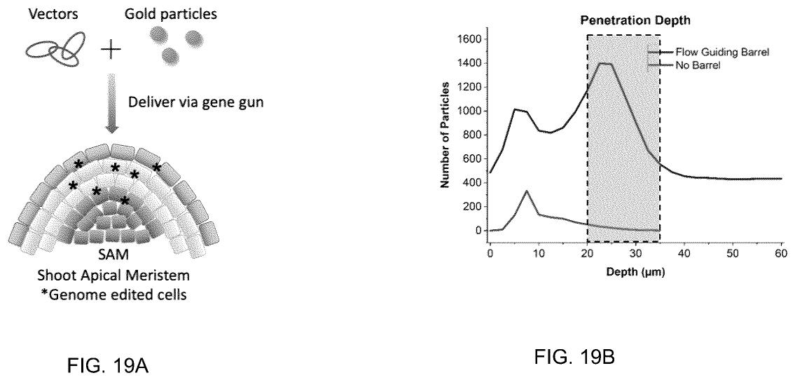

More specifically, in SAM 16 delivery, the microcarriers 180 need to be delivered into the L2 layer 204 of the SAM 16 in order to obtain germline transmittable genetic modifications. With extender 14 improved biolistic particle delivery system 10, the penetration depth 216 of the gold particles or microcarriers 180 can ensure efficient reach of the delivery cargos into the L2 layer 204, as shown in FIG. 19A.

When utilizing extender 14, the penetration depth 216 of microcarriers 180 into L2 layer cells 204 of SAM 16 can be increased by over 4 times compared to the conventional biolistic particle delivery assembly-based SAM delivery method. An increase in penetration depth 216 is supported by the data shown in FIGS. 18B and 19B. This suggests that it is promising to develop an improved SAM delivery method based on the biolistic particle delivery system 10 incorporating the extender 14.

To test the new genetic material delivery system disclosed herein, the conventional SAM 16 delivery method was compared with the extender 14 aided SAM delivery system 10 disclosed herein, by delivering GFP plasmids, or microcarriers 180, into the SAM 16 of wheat. The following describes various testing conducted on the genetic material delivery system disclosed herein and therefore describes such delivery system. The following information is offered by way of illustration and is not intended to limit the invention in any manner. Standard techniques well known in the art or the techniques specifically described below were utilized.

The conventional delivery method has high general GFP-DNA delivery efficiency with 3 shots, a 6 cm flying distance, and 1350 psi rupture disks. When the extender 14 is attached to the biolistic particle delivery assembly 12, precise penetration of the L2 layer cells 204 is achieved by using extender 14 at lower rupture disk pressures (650 psi, 900 psi), with only one shot and two different flying distances (6 cm and 9 cm), as shown in FIG. 7B. The low rupture disk pressures may be higher or lower than 650 psi or 900 psi. For example, the low rupture pressure may range from 400-1200 psi, 600-700 psi or 800-1000 psi or any other range that allows for precise penetration of the L2 layer 204. In one or more arrangements, for example, the low rupture pressure may be adjusted based upon one or more factors including but not limited to: the flying distances; the location of the stop screen, the components of the microcarrier 180, the type of seed, or the type of delivery assembly 12 (e.g. whether the delivery assembly is portable). The flow of microcarriers 180 through the channel 138 resulted in a higher GFP delivery efficiency (directly on SAM 16, two times higher) compared to the conventional SAM delivery method. Conventional methods use a gold particle with a thickness of 0.3 to 0.8 μm. The carrier gas 60 is pressurized between 1,100 to 1,600 psi in order to facilitate bombardment of the microcarriers 180. The microcarriers 180 are propelled at a particle flight distance 104 is 6 cm or less. In order to achieve penetration of the L2 layer cells 204, the system 10 bombards the target cells 218 at least twice.

Condition 1 in the table below, utilizes conventional bombardment methods and used 0.6 μm gold particles (Bio-Rad), with a 6.0 cm flying distance (from stop screen to plate), 1,350 psi pressure, and a 6 mm macrocarrier travel distance. Each Petri dish was bombarded three times. Each shot delivered 1.5 μg of plasmids (1.5 μl, 1 μg/μl) and 300 μg of gold particles (3 μl, 100 μg/μl). Bombardment was conducted using a PDS-1000/He™ device (Bio-Rad, USA). When using the extender 14, only a single bombardment per Petri dish was performed. When an extender 14 is used as part of system 12 a gold particle with a thickness of 0.6 μm may be used. The carrier gas is pressurized at 650 psi in order to facilitate bombardment of the microcarriers 180. The microcarriers 180 are propelled at a particle flight distance 104 is 6 cm to 9 cm. In order to achieve penetration of the L2 layer cells 204, it takes one shot as shown in Table 1.

TABLE 1

| | Conventional Methods | System 10 |
|---|---|---|
| Tissue (shoot | embryo of a fully mature seed; | embryo of a fully mature seed; |
| apex; shoot apical | young bud of a tuber; | young bud of a tuber; |
| meristem) | terminal bud or a lateral bud; | terminal bud or a lateral bud; |
| crops | wheat, barley, rice, corn, soybean, | Wheat, einkorn, corn, soybean |
| | potato, and apple | and other plant species |
| Biolistic delivery | Gold particle: 0.3 to 0.8 μm; | Gold particle: 0.6 μm; |
| condition | Pressure: 1,100 to 1,600 psi; | Pressure: 650 psi; |
| | Flying distance: 6 cm or less; | Flying distance: 6 cm to 9 cm; |
| | Shots: at least two; | Shots: one shot; |
| | | With Extender 14 |
| vectors | At least one nucleic acid: linear DNA | An optimized vector, which is |
| | is a linear plasmid and further | bigger than 1.2 kb. |
| | comprises 0.8 to 1.2 kb-long nucleic | |
| | acids | |

With extender 14 aided SAM 16 delivery, there is at least two times increase in GFP expression efficiency when tested in SAM 16 cells (L1 layer 202 or L2 layer cells 204) at lower gas pressure, only one shot and two different flying distances (6 cm and 9 cm) compared to conventional SAM 16 delivery method with 3 shots. The conventional SAM delivery method and the extender 14-aided SAM 16 delivery methods were further compared, by delivering ttLbCas12a-PDS construct (targeted on TaPDS gene in wheat) into the SAM 16 of wheat. Several conditions were tested to compare GFP delivery efficiency between the previously reported conditions and bombardment with the newly designed focused gene bombardment system 10 aided by extender 14, as shown for example in Table 2). Conventional bombardment conditions (Condition 1) were based on previous studies, employing the 1350 psi pressure, 6 cm flight distance, and three shots. Using condition 1, we observed an overall GFP delivery efficiency of 93%; however, the efficiency of SAM 16-specific delivery was only 17%, as shown in Table 2. Coupling the conventional pressure and flight distance with one shot, only 3% GFP delivery was achieved with no GFP expression in SAM 16 tissue as shown in Table 2. In contrast, conditions 3, 4, and 5 utilizing system 10 resulted in 100% GFP delivery efficiency, with SAM 16-specific delivery reaching 33%, 53%, and 17%, respectively as shown in Table 2. Condition 3 and 4 exhibited twice the SAM 16-specific delivery efficiency of the conventional method as shown in Table 2. Notably, more GFP dots in SAM cells were observed using Condition 3 and 4 compared to Condition 1, indicating enhanced SAM-specific delivery and expression. This data suggests that with only one shot, system 10 aided delivery utilizing extender 14 resulted higher efficiency of wheat SAM 16 transformation, albeit a low gas pressure was used. Conditions 3 and 4 consistently demonstrated higher overall and SAM 16-specific GFP delivery efficiencies. The delivery efficiency was calculated as the ratio of positive SAMs 16 to the total number of bombarded SAMs 16. The GFP fluorescence was detected 12 hours after bombardment and embryos carrying 10 or more GFP signal spots were considered GFP-positive. The GFP fluorescence was detected 12 hours after bombardment and embryos whose SAM 16 (L1/L2/L3 layer) cells showing GFP signals were counted as positive.

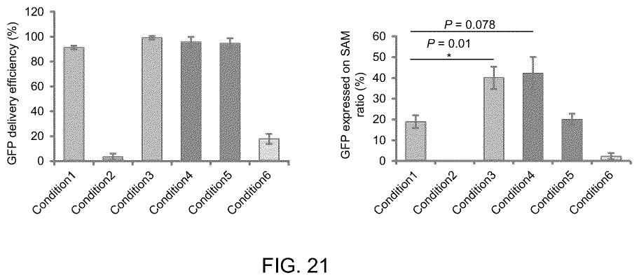

The results show that the propulsion of microcarriers 180 through channel 138 causes more precise delivery of the microcarriers 180 to the target cells 218 and can achieve a two times increase in genome editing (GE) efficiency in TO generation. Further, these results are also at lower gas pressure and only use one shot and two different flying distance (6 cm and 9 cm) as shown in Table 2 below and FIG. 21.

TABLE 2

| | | | | | GE | GFP expressed |
|---|---|---|---|---|---|---|
| | | FGB | Flying | | efficiency | in SAM |
| | Shot | (Y/N) | distance | psi | in T0 | cells ratio |
| Condition 1 | 3 | N | 6 cm | 1350 | 93% | 17% |
| Condition 2 | 1 | N | 6 cm | 1350 | 3% | 0% |
| Condition 3 | 1 | Y | 6 cm | 650 | 8.7% | 33% |
| Condition 4 | 1 | Y | 9 cm | 650 | 9.3% | 53% |
| Condition 5 | 1 | Y | 6 cm | 900 | 4% | 17% |
| Condition 6 | 1 | Y | 9 cm | 900 | 13% | 3% |

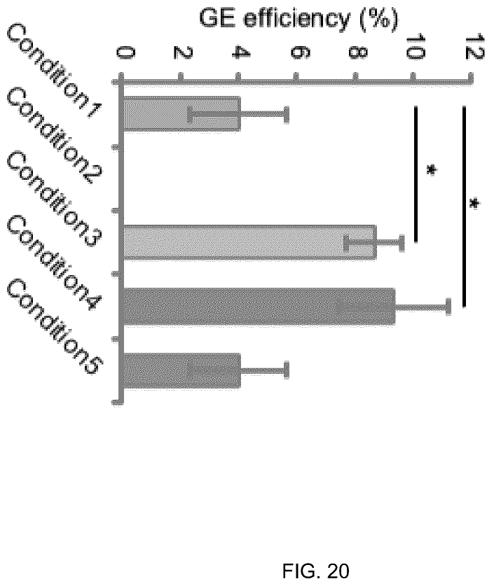

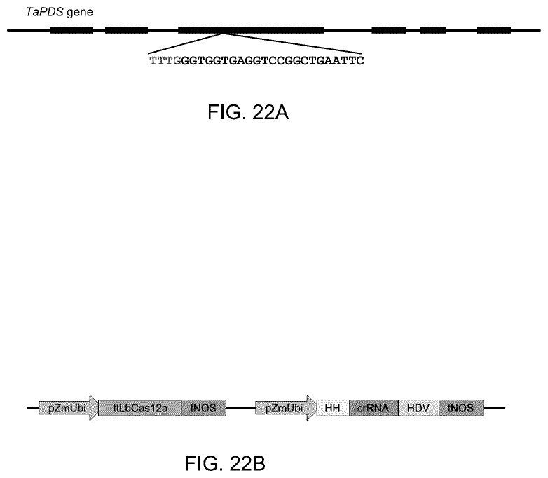

Given that SAM 16-specific GFP delivery efficiency was significantly improved with use of the extender 14/system 10, extender 14/system 10 may also enhance the delivery efficiency of CRISPR-based genome editing tools, thereby increasing SAM 16-based genome editing efficiency in wheat. In one or more arrangements a CRISPR-Cas12a mediated genome editing system 10 based on SAM 16 delivery may be utilized, because prior SAM 16-delivery based genome editing focused on CRISPR-Cas9 and Agrobacterium-mediated CRISPR-Cas12a delivery resulted in extremely low editing efficiency in wheat. In one or more arrangements, the TaPDS gene (present in the A, B and D sub-genomes) was used as the target for Cas12a-mediated editing, as shown in FIG. 22A. The target construct included ttLbCas12a⁷ and the crRNA, both driven by the ZmUbi promoter, as shown in FIG. 22B. To determine the optimal experimental conditions for achieving the highest genome editing (GE) efficiency through SAM 16-based delivery, five different conditions with three independent experiments were evaluated, as shown in FIG. 20. Condition 1 resulted in an average GE efficiency of 4.0% in the TO generation, and no positive TO plants were obtained under condition 2. In contrast, Conditions 3 and 4 achieved significantly higher GE efficiencies of 8.7% and 9.3% in TO generation, respectively. Condition 5 yielded a GE efficiency of 4.0% in the TO generation. As such, the use of the extender 14 in system 10 enhances the efficiency of SAM 16-based CRISPR-Cas12a-mediated genome editing two times or more.

Additionally, in one or more arrangements, in operation, for example, all positive TO plants were propagated, their seeds harvested, and germinated to assess whether the mutations were transmitted to the T1 generation. Under condition 1, a GE efficiency of 27.8% was observed in the T1 generation. In contrast, GE efficiencies of 45.0% and 55.6% were achieved in the T1 generation under conditions 3 and 4, respectively. Condition 5 resulted in a GE efficiency of 44.4% in the T1 generation. The genotype of all T1 plants was analyzed, this confirmed that the mutations were successfully transmitted from the parent progeny (TO). These results indicate that the system 10 and the extender 14 significantly improves conventional SAM 16-based delivery by enhancing the targeting of gold particles to the L2 layer cells 204, thereby increasing the heritability of mutations from the T0 to the T1 generation.

System 10 significantly enhances GE efficiency in wheat by facilitating precise delivery of CRISPR/Cas12a components specifically to the L2 layer cells 204 of SAM 16. This targeted approach increases the likelihood of heritable mutations, improving both the stability and effectiveness of genome modifications. System 10 achieves high SAM 16-specific delivery with fewer bombardments, minimizing physical damage to plant tissues and substantially reducing the consumption of gold particles. This reduction translates into lower operational costs, making system 10 a cost-effective alternative to conventional bombardment methods that typically require multiple shots and higher resource input. By enhancing the efficiency and precision of genome modifications, system 10 and extender 14 expedite the development of crop varieties with desirable traits, thereby contributing to advancements in agricultural biotechnology and food security.

The present disclosed genetic material delivery system is additionally described by reference in the following Examples which are offered by way of illustration and are not intended to limit the disclosed genetic material delivery system in any manner. Standard techniques well known in the art or the techniques specifically described below were utilized.

Einkorn (Triticum monococcum, 2n=2x=14) is a diploid wheat with its genome resembling the AA genome of bread wheat. It is the first domesticated wheat, and its cultivation dates back to the Fertile Crescent around 8000 BC. Recently, select wild and domesticated einkorn wheat genomes have been sequenced (Ahmed, et al. 2023). Furthermore, a large pan-genome sequencing project on einkorn wheat is underway. The establishment of these genomic resources makes einkorn wheat an attractive system for pre-breeding research and germplasm development. However, a reliable genetic transformation pipeline has not been established in einkorn (Miroshnichenko, et al. 2018), which severely limits research progress in einkorn.

We compared the conventional SAM 16 delivery method with the present extender 14 aided SAM 16 delivery method by introducing GFP plasmids into the SAM 16 of Einkorn. Specifically, we evaluated the conventional bombardment condition against the optimized condition using the extender 14 device. Our results, shown in Table 3, showed that employing the extender 14 at a lower gas pressure (650 psi) achieved a substantially higher (nearly twofold) overall delivery efficiency compared to the conventional SAM 16 delivery method in Einkorn. As shown in the following table, the extender 14-assisted approach consistently produced approximately a twofold increase in GFP expression efficiency in SAM 16 cells (L1 layer 202 or L2 layer 204) at the lower gas pressure, relative to the conventional method. These findings indicate that this new delivery strategy enables robust and highly efficient DNA delivery of GFP constructs into the L2 layer 204 of the SAM 16.

TABLE 3

| | | | | | | GFP delivery efficiency | | | GFP-on-SAM efficiency | | |
|---|---|---|---|---|---|---|---|---|---|---|---|
| | | | | Flying | Gas | GFP | | | Right | | |
| | | | | distance | pressure | positive | | Average ± | on | | Average ± |
| Method | Exp | SAM | Shot | (cm) | (psi) | SAM* | Ratio*** | sd (%) | SAM* | Ratio*** | SD (%) |
| conventional | 1st | 30 | 3 | 6 | 1350 | 14 | 47% | 48 ± 2 | 6 | 20% | 16 ± 4 |
| | 2nd | 30 | 3 | 6 | 1350 | 14 | 47% | | 5 | 17% | |
| | 3rd | 30 | 3 | 6 | 1350 | 15 | 50% | | 3 | 10% | |
| with FGB | 1st | 32 | 1 | 6 | 650 | 29 | 91% | 87 ± 6 | 9 | 28% | 30 ± 2 |
| | 2nd | 32 | 1 | 6 | 650 | 29 | 91% | | 9 | 28% | |
| | 3rd | 30 | 1 | 6 | 650 | 24 | 80% | | 10 | 33% | |

*After bombardment, if SAM 16 have over 10 GFP dots counted as GFP positive SAM 16.
** After bombardment, if SAM 16 have GFP dots right on SAM 16 cells counted as right on SAM 16.
***The delivery efficiency was calculated as the ratio of positive SAMs 16 to the total number of bombarded SAMs 16.
10

Next-generation sequencing (NGS) may be utilized to analyze genome editing outcomes of delivering genetic material utilizing system 10. In one or more arrangements, for example, ror positive plant screening, the targeted genomic regions were directly amplified from 5^{th} leaf tissue of T0 wheat plants following the instructions of Phire Plant Direct PCR Kit (Thermo Fisher) and barcoded using the Hi-TOM primers. PCR products were verified by gel electrophoresis. PCR products were pooled together and purified with QIAQuick PCR Purification Kit (QIAGEN) and then quantified by Nanodrop (Thermo Fisher). The purified PCR products were used for Illumina HiSeq2500 sequencing. For targeted mutagenesis, the NGS data were analyzed with CRISPRMatch and CRISPResso2. Given the chimeric nature of the SAM 16-based bombardment, the mutation ratio over 2% will be treated as positive plants in TO generation. The NGS data also used to detect the mutations of genome type by using specific sequence of A, B and D genome in wheat. NGS was also used to detect the genotype of positive T1 wheat plants. These edited T1 wheat plants were categorized into three genotypes including homozygous, heterozygous, and wild-type following these criteria: homozygous, one type of mutation≥70%; heterozygous, one type of mutation>30% but total mutation frequency<70%; wild-type, total mutation frequency<30%. Table 4 shows a comprehensive summary of the experimental results evaluating SAM 16-based GE efficiency, comparing delivery with and without the use of system 10 or extender 14. Three independent experiments were conducted. The number of positive TO and T1 plants was recorded in each individual experiment. The GE efficiency in TO generation was calculated as the ratio of average positive TO plants to the total number of bombarded SAMs 16. For T1 plants, those derived from the same TO plant were counted as one positive T1 event. The GE efficiency in T1 generation was calculated as the ratio of average positive T1 plants to the total number of positive T0 plants.

TABLE 4

| | Bom- | | | | | | | | T0 GE | | | | T1 GE |
|---|---|---|---|---|---|---|---|---|---|---|---|---|---|
| | barded | | FGB | Flying | | Positive T0 | | | efficiency (%) | Positive T1 | | | efficiency (%) |
| | SAM | Shot | (Y/N) | distance | Psi | Ex1 | Ex2 | Ex3 | Average ± sd | Ex1 | Ex2 | Ex3 | Average ± sd |
| Condition 1 | 50 | 3 | N | 6 cm | 1350 | 1 | 3 | 2 | 4.0 ± 1.6 | 0 | 1 | 1 | 27.8 ± 20.8 |
| Condition 2 | 50 | 1 | N | 6 cm | 1350 | 0 | 0 | 0 | 0.0 ± 0.0 | 0 | 0 | 0 | 0.0 ± 0.0 |
| Condition 3 | 50 | 1 | Y | 6 cm | 650 | 4 | 5 | 4 | 8.7 ± 0.9 | 1 | 3 | 2 | 45.0 ± 14.7 |
| Condition 4 | 50 | 1 | Y | 9 cm | 650 | 4 | 6 | 4 | 9.3 ± 1.9 | 3 | 4 | 1 | 55.6 ± 21.9 |
| Condition 5 | 50 | 1 | Y | 6 cm | 900 | 2 | 1 | 3 | 4.0 ± 1.6 | 0 | 1 | 1 | 44.4 ± 41.6 |

TABLE 5

| | | | | | | | General GFP delivery efficiency | | | | | |
|---|---|---|---|---|---|---|---|---|---|---|---|---|
| | | | | | | | GFP | | | GFP-on-SAM efficiency | | |
| | | | | FGB | Flying | | positive | | Average ± | Right on | | Average ± |
| Group | Exp | SAM | Shot | (Y/N) | Distance | psi | SAM* | Ratio*** | sd (%) | SAM** | Ratio*** | sd (%) |
| Condition 1 | 1st | 30 | 3 | N | 6 cm | 1350 | 28 | 93% | 91 ± 2 | 5 | 17% | 19 ± 3 |
| | 2nd | 30 | 3 | N | 6 cm | 1350 | 27 | 90% | | 7 | 23% | |
| | 3rd | 30 | 3 | N | 6 cm | 1350 | 27 | 90% | | 5 | 17% | |
| Condition 2 | 1st | 30 | 1 | N | 6 cm | 1350 | 1 | 3% | 3 ± 2 | 0 | 0% | 0 ± 0 |
| | 2nd | 30 | 1 | N | 6 cm | 1350 | 0 | 0% | | 0 | 0% | |
| | 3rd | 30 | 1 | N | 6 cm | 1350 | 2 | 7% | | 0 | 0% | |
| Condition 3 | 1st | 30 | 1 | Y | 6 cm | 650 | 30 | 100% | 99 ± 2 | 10 | 33% | 40 ± 5 |
| | 2nd | 30 | 1 | Y | 6 cm | 650 | 29 | 97% | | 14 | 47% | |
| | 3rd | 30 | 1 | Y | 6 cm | 650 | 30 | 100% | | 12 | 40% | |
| Condition 4 | 1st | 30 | 1 | Y | 9 cm | 650 | 30 | 100% | 96 ± 4 | 16 | 53% | 42 ± 8 |
| | 2nd | 30 | 1 | Y | 9 cm | 650 | 27 | 90% | | 11 | 37% | |
| | 3rd | 30 | 1 | Y | 9 cm | 650 | 29 | 97% | | 11 | 37% | |
| Condition 5 | 1st | 30 | 1 | Y | 6 cm | 900 | 30 | 100% | 94 ± 4 | 5 | 17% | 20 ± 3 |
| | 2nd | 30 | 1 | Y | 6 cm | 900 | 28 | 93% | | 7 | 23% | |
| | 3rd | 30 | 1 | Y | 6 cm | 900 | 27 | 90% | | 6 | 20% | |
| Condition 6 | 1st | 30 | 1 | Y | 9 cm | 900 | 4 | 13% | 18 ± 4 | 1 | 3% | 2 ± 2 |
| | 2nd | 30 | 1 | Y | 9 cm | 900 | 7 | 23% | | 0 | 0% | |
| | 3rd | 30 | 1 | Y | 9 cm | 900 | 5 | 17% | | 1 | 3% | |
| Condition 7 | 1st | 30 | 1 | Y | 6 cm | 1350 | 0 | 0% | 0 | 0 | 0% | 0 |
| Condition 8 | 1st | 30 | 1 | N | 6 cm | 650 | 0 | 0% | 0 | 0 | 0% | 0 |
| | 2nd | 30 | 1 | N | 6 cm | 650 | 0 | 0% | | 0 | 0% | |

The above Table 5 shows a detailed summary of GFP delivery efficiency into SAM 16, with and without the use of system 10.

TABLE 6

| | | | | | | | | | | T0 GE | | | T1 GE |
|---|---|---|---|---|---|---|---|---|---|---|---|---|---|
| | | | | | | | | T0 | | efficiency | T1 | | efficiency |
| | | | | FGB | Flying | | T0 | positive | T0 GE | average ± | positive | T1 GE | average ± |
| Group | Exp | SAM | Shot | (Y/N) | Distance | psi | plants | plants | efficiency | sd (%) | plants | efficiency | sd (%) |
| Condition 1 | 1st | 50 | 3 | N | 6 cm | 1350 | 45 | 1 | 2% | 4.0 ± 1.6 | 0 | 0% | 27.8 ± 20.8 |
| | 2n | 50 | 3 | N | 6 cm | 1350 | 45 | 3 | 6% | | 1 | 2% | |
| | 3rd | 50 | 3 | N | 6 cm | 1350 | 34 | 2 | 4% | | 1 | 2% | |
| Condition 2 | 1st | 50 | 1 | N | 6 cm | 1350 | 42 | 0 | 0% | 0 ± 0 | 0 | 0% | 0 ± 0 |
| | 2nd | 50 | 1 | N | 6 cm | 1350 | 44 | 0 | 0% | | 0 | 0% | |
| | 3rd | 50 | 1 | N | 6 cm | 1350 | 35 | 0 | 0% | | 0 | 0% | |
| Condition 3 | 1st | 50 | 1 | Y | 6 cm | 650 | 48 | 4 | 8% | 8.7 ± 0.9 | 1 | 2% | 45.0 ± 14.7 |
| | 2n | 50 | 1 | Y | 6 cm | 650 | 47 | 5 | 10% | | 3 | 6% | |
| | 3rd | 50 | 1 | Y | 6 cm | 650 | 43 | 4 | 8% | | 2 | 4% | |
| Condition 4 | 1st | 50 | 1 | Y | 9 cm | 650 | 47 | 4 | 8% | 9.3 ± 1.9 | 3 | 6% | 55.6 ± 21.9 |
| | 2n | 50 | 1 | Y | 9 cm | 650 | 40 | 6 | 12% | | 4 | 8% | |
| | 3rd | 50 | 1 | Y | 9 cm | 650 | 50 | 4 | 8% | | 1 | 2% | |
| Condition 5 | 1st | 50 | 3 | Y | 6 cm | 650 | 47 | 2 | 4% | 4.0 ± 1.6 | 0 | 0% | 44.4 ± 41.6 |
| | 2nd | 50 | 3 | Y | 6 cm | 650 | 45 | 1 | 2% | | 1 | 2% | |
| | 3rd | 50 | 3 | Y | 6 cm | 650 | 36 | 3 | 6% | | 1 | 2% | |

The above Table 6 shows a detailed summary of the experimental results evaluating SAM 16-based genome editing (GE) efficiency, comparing delivery with and without using system 10.

Tables 7-10 are representative screening results of edited TO plants genotype of T1 plants generated under different delivery conditions. The PAM sequence is TTTG (as shown in SEQ ID NO: 1) while the wild-type alleles sequence is GGTGGTGAGGTCCGGCTGAATTC (as shown in SEQ ID NO: 2). Allelic variants were quantified using Hi-TOM next-generation sequencing (NGS).

TABLE 7
Targeted Gene Information

| | TraesCS4D02G299000; TraesCS4A02G004900; | |
|---|---|---|
| TapPDS gene ID | TraesCS4B02G300100 (IWGSC) | |
| TaPDS crRNA | TTTGGGTGGTGAGGTCCGGCTGAATTC (as | sequence 5′-3′ |
| sequence | shown in SEQ ID NO: 3) | |
| Amplicon of target | AAATGTGTAGGAGAAGCATGGCTCGAAAATG | |
| region | GCATTCTTGGATGGTAATCCTCCTGAAAGGCT | |
| | ATGCATGCCTATTGTTAACCACATTCAGTCTTT | |
| | GGGTGGTGAGGTCCGGCTGAATTCTCGTATTC | |
| | AGAAAATTGAACTGAACCCTGACGGAACTGT | |
| | GAAGCACTTTGCACTTACTGATGGGACTCAAA | |
| | TAACTGGAGATGCATATGTTTGTGCAGCACCA | |
| | GGTGCGATTT (as shown in SEQ ID NO: 4) | |

TABLE 8
A, B, and D genome specific sequence of TaPDS gene

| | | |
|---|---|---|
| sequence_A_F | GTAATCCTCCTGAAAGGCTATGCA (as | A, B, and D genome |
| | shown in SEQ ID NO: 5) | specific sequence of |
| sequence_A_R | GGACGGAACAGTGAA (as shown in | TaPDS gene, to |
| | SEQ ID NO: 6) | distinguish sequence |
| sequence_B_F | GTAATCCTCCTGAAAGGCTATGCA (as | results. |
| | shown in SEQ ID NO: 7) | |
| sequence_B_R | TGACGGAACAGTGAA (as shown in | |
| | SEQ ID NO: 8) | |
| sequence_D_F | GTAATCCTCCTGAAAGGCTATGCA (as | |
| | shown in SEQ ID NO: 9) | |
| sequence_D_R | TGACGGAACTGTGAA (as shown in | |
| | SEQ ID NO: 10) | |

TABLE 9

| | T0 | | | | Ratio |
|---|---|---|---|---|---|
| | lines | Genome | Aligned Sequence | | (%) |
| | Ref | | GTCTTTGGGTGGTGAGGTCCGGCTGAATTCTCGTATTCAG | SEQ ID | |
| | | | | NO: 11 | |
| Condition 1 | C1-2 | B | GTCTTTGGGTGGTGAGGTC-----------TCGTATTCAG | SEQ ID | 3.72 |
| | | | | NO: 12 | |
| Condition 3 | C3-2 | B | GTCTTTGGGTGGTGAGGTC-----------TCGTATTCAG | SEQ ID | 2.69 |
| | | | | NO: 13 | |
| | C3-11 | B | GTCTTTGGGTG-------------------TCGTATTCAG | SEQ ID | 3.76 |
| | | | | NO: 14 | |
| | | D | GTCTTTGGGTG-------------------TCGTATTCAG | SEQ ID | 4.53 |
| | | | | NO: 15 | |
| | C3-13 | A | GTCTTTGGGTGGTGAGGTCCG---------TCGTATTCAG | SEQ ID | 2.64 |
| | | | | NO: 16 | |
| Condition 4 | C4-5 | D | GTCTTTGGGTG-------------------TCGTATTCAG | SEQ ID | 2.60 |
| | | | | NO: 17 | |
| | C4-8 | B | GTCTTTGGGTGGTGAGGTCCG-------TCTCGTATTCAG | SEQ ID | 3.28 |
| | | | | NO: 18 | |
| | C4-9 | D | GTCTTTGGGTG-------------------TCGTATTCAG | SEQ ID | 3.73 |
| | | | | NO: 19 | |
| | C4-10 | A | GTCTTTGGGTGGTGAGGTCCG---------TCGTATTCAG | SEQ ID | 3.72 |
| | | | | NO: 20 | |
| Condition 5 | C5-3 | B | GTCTTTGGGTGGTGAGGTCCG--------CTCGTATTCAG | SEQ ID | 3.46 |
| | | | | NO: 21 | |

TABLE 10

| | T1 | | | | Ratio | Geno- |
|---|---|---|---|---|---|---|
| | lines | Genome | Aligned Sequence | | (%) | type |
| | Ref | | GTCTTTGGGTGGTGAGGTCCGGCTGAATTCTCGTATTCAG | SEQ ID | | |
| | | | | NO: 22 | | |
| Condition 1 | C1-2-6 | B | GTCTTTGGGTGGTGAGGTC-----------TCGTATTCAG | SEQ ID | 89.22 | AAbbDD |
| | | | | NO: 23 | | |
| Condition 3 | C3-1-4 | D | GTCTTTGGGTG-------------------TCGTATTCAG | SEQ ID | 90.22 | AABBdd |
| | | | | NO: 24 | | |
| | C3-9-1 | B | GTCTTTGGGTGGTGAGGTCCG--------CTCGTATTCAG | SEQ ID | 37.88 | AABbDD |
| | | | | NO: 25 | | |
| | C3-11- | B | GTCTTTGGGTG-------------------TCGTATTCAG | SEQ ID | 79.91 | AAbbdd |
| | 8 | | | NO: 26 | | |
| | | D | GTCTTTGGGTG-------------------TCGTATTCAG | SEQ ID | 85.24 | |
| | | | | NO: 27 | | |
| | C3-11- | B | GTCTTTGGGTG-------------------TCGTATTCAG | SEQ ID | 38.90 | AABbdd |
| | 13 | | | NO: 28 | | |
| | | D | GTCTTTGGGTG-------------------TCGTATTCAG | SEQ ID | 83.10 | |
| | | | | NO: 29 | | |
| Condition 4 | C4-2-7 | A | GTCTTTGGGTGGTGAGGTCCG---------TCGTATTCAG | SEQ ID | 85.37 | aaBBDD |
| | | | | NO: 30 | | |
| | C4-8-5 | B | GTCTTTGGGTGGTGAGGTCCG-------TCTCGTATTCAG | SEQ ID | 86.95 | AAbbDD |
| | | | | NO: 31 | | |
| | C4-10- | A | GTCTTTGGGTGGTGAGGTCCG---------TCGTATTCAG | SEQ ID | 88.54 | aaBBDD |
| | 1 | | | NO: 32 | | |
| | C4-10- | A | GTCTTTGGGTGGTGAGGTCCG---------TCGTATTCAG | SEQ ID | 38.70 | AaBBDD |
| | 2 | | | NO: 33 | | |
| Condition 5 | C5-3-6 | B | GTCTTTGGGTGGTGAGGTCCG--------CTCGTATTCAG | SEQ ID | 47.85 | AABbDD |
| | | | | NO: 34 | | |

This new method resulted in decreased collateral damage to the plant tissue being transformed by the new delivery method due to the lower rupture disk pressure used, which may contribute to the dramatic improvement in transformation efficacy. Furthermore, this method also allows the delivery of genes and proteins into plants that have been recalcitrant to the delivery to the L2 layer 204 of the SAM 16.

It will be appreciated by those skilled in the art that other various modifications could be made to the device without parting from the spirit and scope of this disclosure. All such modifications and changes fall within the scope of the claims and are intended to be covered thereby.

### REFERENCE NUMBERS

10—delivery system (system)
12—biolistic particle delivery assembly
14—extender (“FGB”)
16—shoot apical meristem (“SAM”)
18—microcarrier system
20—control system
30—microcarrier launch assembly
32—chamber
34—shelf (assembly)
36—target shelf
38—container
40—top of assembly shelf
42—bottom of assembly shelf
44—opening of assembly shelf
46—rupture disk retaining cap
48—diameter of opening of assembly shelf
50—top section of chamber
52—bottom section of chamber
54—gas acceleration tube
56—rupture disk
58—opposing sides of the chamber
60—gas
64—recess
66—opposing sides of microcarrier launch assembly
68—opening of gas acceleration chamber
70—portable gene gun
72—cartridges
74—cylinder lock
76—barrel pin
78—cartridge holder
80—o rings (not shown)
82—cylinder advance lever
84—barrel liner
88—trigger button
96—power source (not shown)
98—gas attachment
104—particle flight distance
106—particle flight width
110—retaining ring
112—opening retaining ring
118—top of retaining ring
120—bottom of retaining ring
122—side/opposing sides of retaining ring
124—interior of retaining ring
126—exterior of retaining ring
128—body
130—top of body
132—bottom of body
134—opening in top of body
136—opening in bottom of body
138—channel in body
140—interior of body
142—exterior of body
144—thickness of retaining ring
146—length of extender
148—flow paths of body
150—dividers
154—diameter of channel
156—diameter of body
158—diameter of retaining ring
160—barrel
162—side/opposing sides of body
168—sides of stop screen (not shown)
170—stop screen
172—width of stop screen (not shown)
174—height of stop screen (not shown)
176—top of stop screen (not shown)
178—sample
180—microcarriers
182—plasmids
184—coating
186—genetic material
188—proteins
190—macrocarriers (not shown)
192—bottom of stop screen (not shown)
200—mature seed
202—L1 layer
204—L2 layer
206—L3 layer
208—germline lineage cells
210—plant
212—first generation
214—second generation
216—penetration depth
218—target cells
220—gas containers
222—power mechanism,
224—vacuum switch
226—vacuum gauge
228—vacuum rate valve
230—vacuum control valve
232—gas flow switch
234—gas pressure gauge
236—gas tube
250—placement guide
252—top of placement guide
254—bottom of placement guide
256—sides of placement guide
258—holes of placement guide

What is claimed:

1. A delivery system for delivering genetic material to a shoot apical meristem, the delivery system comprising:
a biolistic particle delivery assembly;
the biolistic particle delivery assembly having a gas acceleration tube;
the biolistic particle delivery assembly having a bombardment chamber;
the bombardment chamber having a microcarrier launch assembly comprising an opening adapted to receive gas for acceleration of a microcarrier system and to direct gas to a target;
a source of pressurized carrier gas operatively connected to a first end of the gas acceleration tube; and
an extender;
the extender having a channel configured to fit through the opening;
the channel having a length running from a top end of the extender to a bottom end of the extender;
the channel configured to direct a major flow path of the microcarrier system through the channel and towards target cells;
wherein the length of the extender facilitates the penetration of the microcarrier system to an L2 layer of the shoot apical meristem.

2. The delivery system of claim 1, wherein the microcarrier system comprises gold particles.

3. The delivery system of claim 1, wherein a carrier gas is pressurized at 400-1200 psi.

4. The delivery system of claim 1, wherein the channel has a top end adjacent to the gas acceleration tube.

5. The delivery system of claim 1, wherein the channel has a top end adjacent to the gas acceleration tube;
wherein the pressurized carrier gas propels the microcarrier system out of a bottom end of the channel towards the target cells.

6. The delivery system of claim 1, wherein the channel has a top end adjacent to the gas acceleration tube;
wherein the pressurized carrier gas propels the microcarrier system out of a bottom end of the channel towards the target cells;
wherein the distance between the bottom end of the channel and the target cells comprises a flying distance of the microcarrier system.

7. The delivery system of claim 1, wherein the channel has a top end adjacent to the gas acceleration tube;
wherein the pressurized carrier gas propels the microcarrier system out of a bottom end of the channel towards the target cells;
wherein the distance between the bottom end of the channel and the target cells comprises a flying distance of the microcarrier system;
wherein the flying distance is between 6 cm and 9 cm.

8. The delivery system of claim 1, wherein the biolistic particle delivery assembly is a helium driven gene gun.

9. The delivery system of claim 1, wherein the delivery system uses a single bombardment of the microcarrier system to deliver the microcarrier system to the target cells.

10. The delivery system of claim 1, wherein the delivery system uses a single bombardment of the microcarrier system to deliver the microcarrier system to penetrate the L2 layer of the shoot apical meristem.

11. The delivery system of claim 1, wherein the carrier gas is pressurized at 400-1200 psi;
wherein the pressurized gas decreases collateral damage to plant tissues of the shoot apical meristem.

12. A delivery system for delivering genetic material to shoot apical meristems, the delivery system comprising:
a biolistic particle delivery assembly configured to deliver a microcarrier system at target cells of the shoot apical meristems;
the biolistic particle delivery assembly having a gas tube; and
the biolistic particle delivery assembly having a cartridge holder having an opening;
a source of pressurized carrier gas operatively connected to a first end of the gas tube;
an extender;
the extender having a channel configured to fit through the opening;
the channel configured to direct a major flow path of the microcarrier system through the channel and towards the target cells;
wherein a length of the channel facilitates the penetration of the microcarrier system to an L2 layer of the shoot apical meristem.

13. The delivery system of claim 12, wherein the delivery system is comprised of a target shelf that comprises a placement guide.

14. The delivery system of claim 12, wherein the channel has a top end adjacent to the gas tube opposing a bottom end;
wherein the pressurized carrier gas propels the microcarrier system out of the bottom end of the channel towards the target cells.

15. The delivery system of claim 12, wherein the channel has a top end adjacent to the gas tube opposing a bottom end;
wherein the pressurized carrier gas propels the microcarrier system out of the bottom end of the channel towards the target cells;
wherein the distance between the bottom end of the channel and the target cells comprises a flying distance of the microcarrier system.

16. The delivery system of claim 12, wherein the channel has a top end adjacent to a gas acceleration tube opposing a bottom end;
wherein the pressurized carrier gas propels the microcarrier system out of the bottom end of the channel towards the target cells;
wherein the distance between the bottom end of the channel and the target cells comprises a flying distance of the microcarrier system;
wherein the flying distance is between 6 cm and 9 cm.

17. The delivery system of claim 12, wherein the microcarrier system comprises a microcarrier and a macrocarrier; and
wherein the biolistic particle delivery assembly comprises a stop screen configured to separate the microcarrier from the macrocarrier.

18. The delivery system of claim 12, wherein the delivery system uses a single bombardment of the microcarrier system to deliver the microcarrier system to the target cells.

19. A delivery system extender, the extender comprising:
a ring having a top end, a bottom end, and at least one side; and
the ring having an opening configured to receive a microcarrier system configured to edit a genome of a plant;
the bottom of the ring configured to attach the extender to a biolistic particle delivery assembly;
a body attached to the ring having a top end, a bottom end, and at least one side;
the body having a channel configured to receive pressurized carrier gas carrying the microcarrier system towards target cells of the plant;
wherein a length of the extender facilitates the penetration of the microcarrier system to an L2 layer of the shoot apical meristem.

20. The delivery system of claim 19, wherein the distance between the bottom end of the channel and the target cells comprises a flying distance of the microcarrier system.

21. The delivery system of claim 19, wherein the distance between the bottom end of the channel and the target cells comprises a flying distance of the microcarrier system; wherein the flying distance is between 6 cm and 9 cm.

22. The delivery system of claim 19, wherein the biolistic particle delivery assembly is a helium driven gene gun.

23. The delivery system of claim 19, wherein the delivery system uses a single bombardment of the microcarrier system to deliver the microcarrier system to the target cells.

24. The delivery system of claim 19, wherein the delivery system uses a single bombardment of the microcarrier system to deliver the microcarrier system to penetrate the L2 layer of the shoot apical meristem.

25. A method for delivering genetic material to shoot apical meristems, the method comprising:
attaching an extender to a biolistic particle delivery assembly;
placing a microcarrier system in the biolistic particle delivery assembly;
moving pressurized gas through a gas tube from a gas container to the biolistic particle delivery assembly;
bombarding the microcarrier system through the extender of the biolistic particle delivery assembly towards target cells utilizing the pressurized gas through the extender; and
penetrating an L2 layer of a shoot apical meristem by the microcarrier system.

26. The method of claim 25, wherein the pressurized gas propels the microcarrier system out of a bottom end of a channel of the extender towards the target cells.

27. The method of claim 25, wherein the extender has a channel with a top end opposing a bottom end;
wherein the pressurized gas propels the microcarrier system out of the bottom end of the channel towards the target cells;
wherein the distance between the bottom end of the channel and the target cells comprises a flying distance of the microcarrier system.

28. The method of claim 25, wherein the extender has a channel with a top end opposing a bottom end;
wherein the pressurized gas propels the microcarrier system out of the bottom end of the channel towards the target cells;
wherein the distance between the bottom end of the channel and the target cells comprises a flying distance of the microcarrier system;
wherein the flying distance is between 6 cm and 9 cm.

29. The method of claim 25, wherein the delivery system uses a single bombardment of the microcarrier system to deliver the microcarrier system to the target cells.

30. The method of claim 25, wherein the delivery system uses a single bombardment of the microcarrier system to deliver the microcarrier system to penetrate the L2 layer of the shoot apical meristem.

31. The method of claim 25, further comprising firing a single bombardment of the microcarrier system at the target cells; and
transforming a genome of the target cells utilizing the microcarrier system.

32. The method of claim 25, further comprising:
firing a single bombardment of the microcarrier system at the target cells;
transforming a genome of the target cells utilizing the microcarrier system;
breeding plants with the transformed genome; and
passing the transformed genome to a second generation of the plants.

33. The method of claim 25, further comprising
firing a single bombardment of the microcarrier system at the target cells comprising a plurality of samples; and
transforming a genome of the target cells utilizing the microcarrier system.

34. The method of claim 25, further comprising:
firing a single bombardment of the microcarrier system at the target cells comprising a plurality of samples; and
transforming a genome of the target cells utilizing the microcarrier system;
wherein the plurality of samples is comprised of a first sample having a first genetic material and a second sample having a second genetic material.

35. The method of claim 25, further comprising:
firing a single bombardment of the microcarrier system at the target cells comprising a plurality of samples; and
transforming a genome of the target cells utilizing the microcarrier system;
wherein the plurality of samples is comprised of a first sample having a first protein and a second sample having a second protein.

36. The method of claim 25, further comprising:
firing a single bombardment of the microcarrier system at the target cells comprising a plurality of samples; and
transforming a genome of the target cells utilizing the microcarrier system;
wherein the plurality of samples is comprised of a first sample having a genetic material and a second sample having a protein.

37. The method of claim 25, further comprising:
firing a first bombardment of the microcarrier system at the target cells comprising a first sample;
firing a second bombardment of the microcarrier system at the target cells comprising a second sample; and
transforming a genome of the target cells utilizing the microcarrier system.


SEQUENCE LISTING (34 sequences)

SEQ ID NO: 1 - skipped

SEQ ID NO: 2 - DNA, 23 bp, unidentified
  source 1..23: mol_type = other DNA
ggtggtgaggtccggctgaattc

SEQ ID NO: 3 - RNA, 27 nt, unidentified
  source 1..27: mol_type = other RNA
tttgggtggtgaggtccggctgaattc

SEQ ID NO: 4 - DNA, 233 bp, unidentified
  source 1..233: mol_type = other DNA
aaatgtgtaggagaagcatggctcgaaaatggcattcttggatggtaatcctcctgaaaggctatgcatgcctattgttaaccacattcagtctttgggtggtgaggtccggctgaattctcgtattcagaaaattgaactgaaccctgacggaactgtgaagcactttgcacttactgatgggactcaaataactggagatgcatatgtttgtgcagcaccaggtgcgattt

SEQ ID NO: 5 - DNA, 24 bp, unidentified
  source 1..24: mol_type = other DNA
gtaatcctcctgaaaggctatgca

SEQ ID NO: 6 - DNA, 15 bp, unidentified
  source 1..15: mol_type = other DNA
ggacggaacagtgaa

SEQ ID NO: 7 - DNA, 24 bp, unidentified
  source 1..24: mol_type = other DNA
gtaatcctcctgaaaggctatgca

SEQ ID NO: 8 - DNA, 15 bp, unidentified
  source 1..15: mol_type = other DNA
tgacggaacagtgaa

SEQ ID NO: 9 - DNA, 24 bp, unidentified
  source 1..24: mol_type = other DNA
gtaatcctcctgaaaggctatgca

SEQ ID NO: 10 - DNA, 15 bp, unidentified
  source 1..15: mol_type = other DNA
tgacggaactgtgaa

SEQ ID NO: 11 - DNA, 40 bp, unidentified
  source 1..40: mol_type = other DNA
gtctttgggtggtgaggtccggctgaattctcgtattcag

SEQ ID NO: 12 - DNA, 40 bp, unidentified
  source 1..40: mol_type = other DNA
gtctttgggtggtgaggtcnnnnnnnnnnntcgtattcag

SEQ ID NO: 13 - DNA, 40 bp, unidentified; identical to SEQ ID NO: 12
  source 1..40: mol_type = other DNA

SEQ ID NO: 14 - DNA, 40 bp, unidentified
  source 1..40: mol_type = other DNA
gtctttgggtgnnnnnnnnnnnnnnnnnnntcgtattcag

SEQ ID NO: 15 - DNA, 40 bp, unidentified; identical to SEQ ID NO: 14
  source 1..40: mol_type = other DNA

SEQ ID NO: 16 - DNA, 40 bp, unidentified
  source 1..40: mol_type = other DNA
gtctttgggtggtgaggtccgnnnnnnnnntcgtattcag

SEQ ID NO: 17 - DNA, 40 bp, unidentified; identical to SEQ ID NO: 14
  source 1..40: mol_type = other DNA

SEQ ID NO: 18 - DNA, 40 bp, unidentified
  source 1..40: mol_type = other DNA
gtctttgggtggtgaggtccgnnnnnnntctcgtattcag

SEQ ID NO: 19 - DNA, 40 bp, unidentified; identical to SEQ ID NO: 14
  source 1..40: mol_type = other DNA

SEQ ID NO: 20 - DNA, 40 bp, unidentified; identical to SEQ ID NO: 16
  source 1..40: mol_type = other DNA

SEQ ID NO: 21 - DNA, 40 bp, unidentified
  source 1..40: mol_type = other DNA
gtctttgggtggtgaggtccgnnnnnnnnctcgtattcag

SEQ ID NO: 22 - DNA, 40 bp, unidentified; identical to SEQ ID NO: 11
  source 1..40: mol_type = other DNA

SEQ ID NO: 23 - DNA, 40 bp, unidentified; identical to SEQ ID NO: 12
  source 1..40: mol_type = other DNA

SEQ ID NO: 24 - DNA, 40 bp, unidentified; identical to SEQ ID NO: 14
  source 1..40: mol_type = other DNA

SEQ ID NO: 25 - DNA, 40 bp, unidentified; identical to SEQ ID NO: 21
  source 1..40: mol_type = other DNA

SEQ ID NO: 26 - DNA, 40 bp, unidentified; identical to SEQ ID NO: 14
  source 1..40: mol_type = other DNA

SEQ ID NO: 27 - DNA, 40 bp, unidentified; identical to SEQ ID NO: 14
  source 1..40: mol_type = other DNA

SEQ ID NO: 28 - DNA, 40 bp, unidentified; identical to SEQ ID NO: 14
  source 1..40: mol_type = other DNA

SEQ ID NO: 29 - DNA, 40 bp, unidentified; identical to SEQ ID NO: 14
  source 1..40: mol_type = other DNA

SEQ ID NO: 30 - DNA, 40 bp, unidentified; identical to SEQ ID NO: 16
  source 1..40: mol_type = other DNA

SEQ ID NO: 31 - DNA, 40 bp, unidentified; identical to SEQ ID NO: 18
  source 1..40: mol_type = other DNA

SEQ ID NO: 32 - DNA, 40 bp, unidentified; identical to SEQ ID NO: 16
  source 1..40: mol_type = other DNA

SEQ ID NO: 33 - DNA, 40 bp, unidentified; identical to SEQ ID NO: 16
  source 1..40: mol_type = other DNA

SEQ ID NO: 34 - DNA, 40 bp, unidentified; identical to SEQ ID NO: 21
  source 1..40: mol_type = other DNA